SEQUENCE LISTING (47 sequences)

SEQ ID NO: 1 - DNA, 24 bp, Artificial Sequence
  Forward Primer for HIV
agagaaagagcagaagacagtggm

SEQ ID NO: 2 - DNA, 24 bp, Artificial Sequence
  Forward Primer for HIV
agagaaagagcagaagacagtggc

SEQ ID NO: 3 - DNA, 24 bp, Artificial Sequence
  Forward Primer for HIV
agagaaagagcagaagacagtgga

SEQ ID NO: 4 - DNA, 29 bp, Artificial Sequence
  Reverse Primer for HIV
ccttgtaagtcattggtcttaaaggtacy

SEQ ID NO: 5 - DNA, 29 bp, Artificial Sequence
  Reverse Primer for HIV
ccttgtaagtcattggtcttaaaggtacc

SEQ ID NO: 6 - DNA, 29 bp, Artificial Sequence
  Reverse Primer for HIV
ccttgtaagtcattggtcttaaaggtact

SEQ ID NO: 7 - DNA, 20 bp, Artificial Sequence
  Forward Primer for HIV
gtacaatgtacacatggaat

SEQ ID NO: 8 - DNA, 20 bp, Artificial Sequence
  Reverse Primer for HIV
aaaattctcctctacartta

SEQ ID NO: 9 - DNA, 20 bp, Artificial Sequence
  Reverse Primer for HIV
aaaattctcctctacaatta

SEQ ID NO: 10 - DNA, 20 bp, Artificial Sequence
  Reverse Primer for HIV
aaaattctcctctacagtta

SEQ ID NO: 11 - DNA, 19 bp, Artificial Sequence
  Reverse Primer for HIV
aaaattctcctccacaatt

SEQ ID NO: 12 - DNA, 21 bp, Artificial Sequence
  Forward Primer for HIV
cagcacagtacartgtacaca

SEQ ID NO: 13 - DNA, 19 bp, Artificial Sequence
  Reverse Primer for HIV
cttctccaattgtcyytca

SEQ ID NO: 14 - DNA, 20 bp, Artificial Sequence
  Forward Primer for HIV
ctgttaaatggyagyctagc

SEQ ID NO: 15 - DNA, 18 bp, Artificial Sequence
  Reverse Primer for HIV
caatttctrggtcycctc

SEQ ID NO: 16 - DNA, 21 bp, Artificial Sequence
  Forward Primer for HIV
cagcacagtacaatgtacaca

SEQ ID NO: 17 - DNA, 21 bp, Artificial Sequence
  Forward Primer for HIV
cagcacagtacagtgtacaca

SEQ ID NO: 18 - DNA, 19 bp, Artificial Sequence
  Reverse Primer for HIV
cttctccaattgtccctca

SEQ ID NO: 19 - DNA, 19 bp, Artificial Sequence
  Reverse Primer for HIV
cttctccaattgtccttca

SEQ ID NO: 20 - DNA, 19 bp, Artificial Sequence
  Reverse Primer for HIV
cttctccaattgtctctca

SEQ ID NO: 21 - DNA, 19 bp, Artificial Sequence
  Reverse Primer for HIV
cttctccaattgtctttca

SEQ ID NO: 22 - DNA, 20 bp, Artificial Sequence
  Forward Primer for HIV
ctgttaaatggcagtctagc

SEQ ID NO: 23 - DNA, 20 bp, Artificial Sequence
  Forward Primer for HIV
ctgttaaatggcagcctagc

SEQ ID NO: 24 - DNA, 20 bp, Artificial Sequence
  Forward Primer for HIV
ctgttaaatggtagtctagc

SEQ ID NO: 25 - DNA, 20 bp, Artificial Sequence
  Forward Primer for HIV
ctgttaaatggtagcctagc

SEQ ID NO: 26 - DNA, 18 bp, Artificial Sequence
  Reverse Primer for HIV
caatttctgggtcccctc

SEQ ID NO: 27 - DNA, 18 bp, Artificial Sequence
  Reverse Primer for HIV
caatttctgggtctcctc

SEQ ID NO: 28 - DNA, 18 bp, Artificial Sequence
  Reverse Primer for HIV
caatttctaggtcccctc

SEQ ID NO: 29 - DNA, 18 bp, Artificial Sequence
  Reverse Primer for HIV
caatttctaggtctcctc

SEQ ID NO: 30 - DNA, 23 bp, Artificial Sequence
  Forward Primer for HIV
agagaaagagcagaagacagtgg

SEQ ID NO: 31 - DNA, 28 bp, Artificial Sequence
  Reverse Primer for HIV
ccttgtaagtcattggtcttaaaggtac

SEQ ID NO: 32 - DNA, 20 bp, Artificial Sequence
  Forward Primer for HIV
aaaattcccctccacaatta

SEQ ID NO: 33 - DNA, 19 bp, Artificial Sequence
  Reverse Primer for HIV
aaaattcccctccacaatt

SEQ ID NO: 34 - DNA, 21 bp, Artificial Sequence
  Forward M13 Primer
ttctggcgtaccgttcctgtc

SEQ ID NO: 35 - DNA, 23 bp, Artificial Sequence
  Reverse M13 Primer
gttttcccagtcacgacgttgta

SEQ ID NO: 36 - DNA, 3010 bp, Human immunodeficiency virus
gtgtggtccatagtaatcatagaatataggaaaatattaagacaaagaaaaatagacaggttaattgatagactaagagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagtatacatataggaccagggagagcattttatgcaacaggagaaataataggagatataagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggat

SEQ ID NO: 37 - DNA, 3010 bp, Human immunodeficiency virus
gtgtggtccatagtaatcatagaatataggaaaatattaagacaaagaaaaatagacaggttaattgatagactaagagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagtatacgtataggaccagggagagcattttatgcaacaggaaaaataataggagatataagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggat

SEQ ID NO: 38 - DNA, 3010 bp, Human immunodeficiency virus
gtgtggtccatagtaatcatagaatataggaaaatattaagacaaagaaaaatagacaggttaattgatagactaagagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccagcaacaatacaagaaaaagtatacatataggaccagggagagcattttatacaacaggaaatataataggagatataagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggat

SEQ ID NO: 39 - DNA, 3010 bp, Human immunodeficiency virus
gtgtggtccatagtaatcatagaatataggaaaatattaagacaaagaaaaatagacaggttaattgatagactaagagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgcataagacccaacaacaatacaagaaaaagtatacatataggaccagggagagcaatttatgcaacaggaggcataataggagatataagacgagcatattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggat

SEQ ID NO: 40 - DNA, 922 bp, Human immunodeficiency virus
attacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagtatacatataggaccagggagagcattttatgcaacaggagaaataataggagatataagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagag

SEQ ID NO: 41 - DNA, 922 bp, Human immunodeficiency virus
attacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagtatacgtataggaccagggagagcattttatgcaacaggaaaaataataggagatataagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagag

SEQ ID NO: 42 - DNA, 105 bp, Human immunodeficiency virus
tgtacaagacccaacaacaatacaagaaaaagtatacatataggaccagggagagcattttatgcaacaggagaaataataggagatataagacaagcacattgt

SEQ ID NO: 43 - DNA, 105 bp, Human immunodeficiency virus
tgtacaagacccaacaacaatacaagaaaaagtatacgtataggaccagggagagcattttatgcaacaggaaaaataataggagatataagacaagcacattgt

SEQ ID NO: 44 - DNA, 105 bp, Human immunodeficiency virus
tgtacaagacccagcaacaatacaagaaaaagtatacatataggaccagggagagcattttatacaacaggaaatataataggagatataagacaagcacattgt

SEQ ID NO: 45 - DNA, 105 bp, Human immunodeficiency virus
tgcataagacccaacaacaatacaagaaaaagtatacatataggaccagggagagcaatttatgcaacaggaggcataataggagatataagacgagcatattgt

SEQ ID NO: 46 - DNA, 105 bp, Human immunodeficiency virus; identical to SEQ ID NO: 42

SEQ ID NO: 47 - DNA, 105 bp, Human immunodeficiency virus; identical to SEQ ID NO: 43

(19) United States
(12) Patent Application Publication
(10) Pub. No.: US 2015/0299814 A1
(43) Pub. Date: Oct. 22, 2015

(54) Reagents and methods for HIV coreceptor tropism genotyping

(71) Applicants: Arejas UZGIRIS, Berkeley, CA (US); Sunil PANDIT, Concord, CA (US); Lance PALMER, Robbinsville, NJ (US)

(72) Inventors: Arejas Uzgiris, Berkeley, CA (US); Sunil Pandit, Concord, CA (US); Lance Palmer, Plainsboro, NJ (US)

(73) Assignee: SIEMENS HEALTHCARE DIAGNOSTICS INC., Tarrytown, NY (US)

(21) Appl. No.: 14/433,245

(22) PCT Filed: Oct. 10, 2013
(86) PCT No.: PCT/US2013/064251

§ 371 Date: Apr. 2, 2015

Related U.S. Application Data
(60) Provisional application No. 61/711,785, filed on Oct. 10, 2012.

(51) Int. Cl. C12Q 1/70

(52) U.S. Cl. CPC C12Q 1/703

(57) ABSTRACT

The present disclosure relates to oligonucleotide sequences for amplification primers and their use in performing nucleic acid amplifications of HIV, in particular regions that encode the V3 region of the env glycoprotein. In some embodiments the primers are used in nested PCR methods for the detection or sequencing of the V3 region of the env glycoprotein. The oligonucleotide sequences are also provided assembled as kits that can be used to detect or sequence the V3 region of the env glycoprotein. Control nucleic acids for use in methods and kits of the present disclosure are also provided.

### BACKGROUND

Acquired Immune Deficiency Syndrome or AIDS is thought to have originated in sub-Saharan Africa during the twentieth century and it is now a global epidemic. At the end of 2004, UNAIDS estimated that nearly 40 million people were infected with HIV, the causative agent of AIDS. The World Health Organization estimated that by 2004 the AIDS epidemic had claimed more than 3 million people and that 5 million people had been infected with HIV in the same year. Currently it is estimated that 28 million people have died from AIDS and that HIV is set to infect 90 million Africans alone, resulting in a minimum estimate of 18 million orphans in the African continent alone.

### SUMMARY

The present disclosure relates to oligonucleotide sequences for amplification primers and their use in performing amplifications of HIV nucleic acid sequences, in particular regions that encode the V3 region of the env glycoprotein. In some embodiments the primers are used in nested PCR methods for the detection or sequencing of the V3 region of the env gene. The oligonucleotide sequences are also provided assembled as kits that can be used to detect or sequence the V3 region of the env glycoprotein.

In some embodiments, isolated oligonucleotide amplification primers are provided that comprise a nucleic acid sequence selected from the group consisting of SEQ. ID NOs. 1-29, complementary sequences thereof, active fragments thereof, and combinations thereof.

In some embodiments, collections of primers for amplifying a V3 region of the HIV env genomic sequence or a portion thereof are provided that comprise primer sets selected from the group consisting of Primer Set 1, Primer Set 2, Primer Set 3, Primer Set 4, Primer Set 5, Primer Set 6, Primer Set 7, and Primer Set 8 wherein Primer Set 1 comprises a forward primer comprising SEQ ID NO: 1, or any active fragment thereof, and a reverse primer comprising SEQ ID NO: 4, or any active fragment thereof, Primer Set 2 comprises a forward primer comprising SEQ ID NO: 7, or any active fragment thereof, and a reverse primer comprising SEQ ID NO: 8 or SEQ ID NO: 11, or any active fragments or combinations thereof, Primer Set 3 comprises a forward primer comprising SEQ ID NO: 12, or any active fragment thereof, and a reverse primer comprising SEQ ID NO: 13, or any active fragment thereof, Primer Set 4 comprises a forward primer comprising SEQ ID NO: 14, or any active fragment thereof, and a reverse primer comprising SEQ ID NO: 15, or any active fragment thereof, Primer Set 5 comprises a forward primer comprising SEQ ID NO: 2 or SEQ ID NO: 3, or any active fragments or combinations thereof, and a reverse primer comprising SEQ ID NO: 5 or SEQ ID NO: 6, or any active fragments or combinations thereof, Primer Set 6 comprises a forward primer comprising SEQ ID NO: 7, or any active fragment thereof, and a reverse primer comprising SEQ ID NO: 9, SEQ ID NO: 10, or SEQ ID NO: 11, or any active fragment or combinations thereof, Primer Set 7 comprises a forward primer comprising SEQ ID NO: 16 or SEQ ID NO: 17, or any active fragments or combinations thereof, and a reverse primer comprising SEQ ID NO: 18, SEQ ID NO: 19, SEQ ID NO: 20, and SEQ ID NO: 21, or any active fragments or combinations thereof, and Primer Set 8 comprises a forward primer comprising SEQ ID NO: 22, SEQ ID NO: 23, SEQ ID NO: 24, or SEQ ID NO: 25, or any active fragments or combinations thereof, and a reverse primer comprising SEQ ID NO: 26, SEQ ID NO: 27, SEQ ID NO: 28, or SEQ ID NO: 29, or any active fragments or combinations thereof. In some embodiments, the forward primer of Primer Set 5 comprises SEQ ID NO: 2 and SEQ ID NO: 3, or any active fragments thereof and the reverse primer of Primer Set 5 comprises SEQ ID NO: 5 and SEQ ID NO: 6, or any active fragments thereof. In some embodiments, the forward primer of Primer Set 6 comprises SEQ ID NO: 7, or any active fragment thereof and the reverse primer of Primer Set 6 comprises SEQ ID NO: 9, SEQ ID NO: 10, and SEQ ID NO: 11, or any active fragments thereof.

In some embodiments, kits for amplifying a V3 region of the HIV env genomic sequence or a portion thereof by nested PCR are provided that comprise a set of outer primers consisting of consisting of Primer Set 1, Primer Set 3, Primer Set 5, or Primer Set 7 and a set of inner primers consisting of Primer Set 2, Primer Set 4, Primer Set 6, or Primer Set 8 as described herein. In some embodiments, at least some of the inner primers set are labeled. In some embodiments, at least some of the inner primers are tagged with tag sequences.

In some embodiments, methods of amplifying a V3 region of the HIV env genomic sequence or a portion thereof in a sample are provided comprising contacting the sample with a set of outer primers consisting of Primer Set 1, Primer Set 3, Primer Set 5, or Primer Set 7 as described herein, submitting the resulting mixture to a first nucleic acid amplification reaction, contacting a product of the first nucleic acid amplification reaction with a set of inner primers consisting of Primer Set 2, Primer Set 4, Primer Set 6, or Primer Set 8 as described herein, and submitting the resulting mixture to a second nucleic acid amplification reaction. In some embodiments, the set of outer primers is Primer Set 1 and the set of inner primers is Primer Set 2. In some embodiments, the set of outer primers is Primer Set 3 and the set of inner primers is Primer Set 4. In some embodiments, the nucleic acid amplification reactions comprises submitting the sample or amplification product to a nucleic acid amplification reaction carried out under suitable amplification conditions and in the presence of suitable amplification reaction reagents. In some embodiments, at least some of the inner primers are labeled. In some embodiments, at least some of the inner primers are tagged with tag sequences. In some embodiments, the tag sequences are M13 tag sequences. In some embodiments, the methods further comprise sequencing a product from the second nucleic acid amplification reaction to detect an HIV env genomic sequence.

In some embodiments, a nucleic acid control (e.g., a vector) comprising a sequence that encodes all or a portion of an HIV Env protein or variant thereof is provided. In some embodiments the nucleic acid comprises DNA. In some embodiments the nucleic acid comprises RNA. In some embodiments, the nucleic acid sequence encodes a protein that is at least 80%, 85%, 90%, 95% or 99% homologous to an HIV Env protein. In some embodiments, the nucleic acid of the HIV Env protein is from (or derived from) the US1 or US2 clone.

In some embodiments, a nucleic acid comprising hybridization sites for at least one Primer Set selected from the group comprising Primer Sets 1-10 is provided. In some embodiments, the hybridization sites are perfect complements for the primers in the one or more Primer Sets. In some embodiments, the nucleic acids comprise hybridization sites for each of the primers in Primer Sets 1-10.

In some embodiments, a nucleic acid that encodes a portion of an HIV Env protein that includes the V3 region is provided. In some embodiments, a nucleic acid comprises a sequence encoding a V3 region that is at least 80%, 85%, 90%, 95% or 99% homologous to a sequence selected from the group consisting of SEQ ID NOS.: 42, 43, 44, 45, 46, and 47 (or an RNA equivalent of any one of these DNA sequences, where an “RNA equivalent of a DNA sequence” as used herein means an RNA sequence that includes a “U” (uracil) instead of each “T” (thymine) within the corresponding DNA sequence).

In some embodiments, a nucleic acid comprising a sequence that encodes all or a portion of a CCR5 coreceptor variant HIV Env protein is provided. In some embodiments, a nucleic acid comprising a sequence that encodes all or a portion of a CXCR4 coreceptor variant HIV Env protein is provided. In some embodiments, the nucleic acid sequence encodes a truncated CCR5 coreceptor variant HIV Env protein. In some embodiments, the nucleic acid sequence encodes a truncated CXCR4 coreceptor variant HIV Env protein.

In some embodiments, a nucleic acid comprising a sequence encoding an HIV Env protein or variant thereof where the sequence is at least 80%, 85%, 90%, 95% or 99% homologous with a sequence selected from the group consisting of SEQ ID NOs.: 36, 37, 38, 39, 40 and 41 (or an RNA equivalent) is provided. In some embodiments, the nucleic acid comprises a sequence selected from the group consisting of SEQ ID NOs.: 42, 43, 44, 45, 46 and 47 (or an RNA equivalent). In some embodiments, a nucleic acid that comprises a sequence selected from the group consisting of SEQ ID NOs.: 36, 37, 38, 39, 40 and 41 (or an RNA equivalent) is provided.

In some embodiments, a nucleic acid that comprises a sequence encoding an HIV Env protein or variant thereof wherein the sequence is at least 80%, 85%, 90%, 95% or 99% homologous with the sequence defined by nucleotides 76 through 2916 of SEQ ID NO.: 36 or 38 (or an RNA equivalent) is provided. In some embodiments, the nucleic acid sequence also comprises SEQ ID NO.: 42 or 44 (or an RNA equivalent). In some embodiments, a nucleic acid comprising the sequence defined by nucleotides 76 through 2916 of SEQ ID NO.: 36 or 38 (or an RNA equivalent) is provided. In some embodiments, a nucleic acid comprising a sequence encoding an HIV Env protein or variant thereof where the sequence is at least 80%, 85%, 90%, 95% or 99% homologous with the sequence defined by nucleotides 76 through 2916 of SEQ ID NO.: 37 or 39 (or an RNA equivalent) is provided. In some embodiments, the nucleic acid sequence also comprises SEQ ID NO.: 43 or 45 (or an RNA equivalent). In some embodiments, a nucleic acid that comprises the sequence defined by nucleotides 76 through 2916 of SEQ ID NO.: 37 or 39 (or an RNA equivalent) is provided.

In some embodiments, mixtures of two or more of nucleic acids (optionally two or more vectors or two or more RNA transcripts), wherein the mixtures comprise a first nucleic acid (optionally, a first vector or RNA transcript) that encodes all or a portion of a CCR5 coreceptor variant HIV Env protein and a second nucleic acid (optionally, a second vector or RNA transcript) that encodes all or a portion of a CXCR4 coreceptor variant HIV Env protein are provided. In some embodiments, the mixtures comprising CCR5 or CXCR4 nucleic acids selected from a group comprising SEQ ID NOs.: 36, 37, 38, 39, 40 or 41 (or an RNA equivalent) or a portion or homolog thereof are provided.

In some embodiments, a mixture comprising a first nucleic acid (optionally, a first vector or RNA transcript) that comprises the sequence of SEQ ID NO.: 36 (or a sequence that is at least 80%, 85%, 90%, 95% or 99% homologous to the sequence of SEQ ID NO.: 36) (or an RNA equivalent) and a second nucleic acid (optionally, a second vector or RNA transcript) that comprises the sequence of SEQ ID NO.: 37 (or a sequence that is at least 80%, 85%, 90%, 95% or 99% homologous to the sequence of SEQ ID NO.: 37) (or an RNA equivalent) is provided. In other embodiments, a mixture comprising a first nucleic acid (optionally, a first vector or RNA transcript) that comprises the sequence defined by nucleotides 76 through 2916 of SEQ ID NO.: 36 (or a sequence that is at least 80%, 85%, 90%, 95% or 99% homologous with the sequence defined by nucleotides 76 through 2916 of SEQ ID NO.: 36) (or an RNA equivalent) and a second nucleic acid (optionally, a second vector or RNA transcript) that comprises the sequence defined by nucleotides 76 through 2916 of SEQ ID NO.: 37 (or a sequence that is at least 80%, 85%, 90%, 95% or 99% homologous with the sequence defined by nucleotides 76 through 2916 of SEQ ID NO.: 37) (or an RNA equivalent) is provided.

In some embodiments, a mixture comprising a first nucleic acid (optionally, a first vector or RNA transcript) that comprises the sequence of SEQ ID NO.: 38 (or a sequence that is at least 80%, 85%, 90%, 95% or 99% homologous to the sequence of SEQ ID NO.: 38) (or an RNA equivalent) and a second nucleic acid (optionally, a second vector) that comprises the sequence of SEQ ID NO.: 39 (or a sequence that is at least 80%, 85%, 90%, 95% or 99% homologous to the sequence of SEQ ID NO.: 39) (or an RNA equivalent) is provided. In other embodiments, a mixture comprising a first nucleic acid (optionally, a first vector) that comprises the sequence defined by nucleotides 76 through 2916 of SEQ ID NO.: 38 (or a sequence that is at least 80%, 85%, 90%, 95% or 99% homologous with the sequence defined by nucleotides 76 through 2916 of SEQ ID NO.: 38) (or an RNA equivalent) and a second nucleic acid (optionally, a second vector) that comprises the sequence defined by nucleotides 76 through 2916 of SEQ ID NO.: 39 (or a sequence that is at least 80%, 85%, 90%, 95% or 99% homologous with the sequence defined by nucleotides 76 through 2916 of SEQ ID NO.: 39) (or an RNA equivalent) is provided.

In some embodiments, a mixture comprising a first nucleic acid (optionally, a first vector or RNA transcript) that comprises the sequence of SEQ ID NO.: 40 (or a sequence that is at least 80%, 85%, 90%, 95% or 99% homologous to the sequence of SEQ ID NO.: 40) (or an RNA equivalent) and a second nucleic acid (optionally, a second vector or RNA transcript) that comprises the sequence of SEQ ID NO.: 41 (or a sequence that is at least 80%, 85%, 90%, 95% or 99% homologous to the sequence of SEQ ID NO.: 41) (or an RNA equivalent) is provided.

In some embodiments, a mixture comprising one of the CCR5 nucleic acids and one of the CXCR4 nucleic acids wherein the molar concentration of the CCR5 nucleic acid is greater than the molar concentration of the CXCR4 nucleic acid (e.g., at least 2, 3, 5, 10, 25, 50 or at least 100 times greater) is provided. In some embodiments, a mixture comprising less than 15, 10, 8, 6, 4, 2, 1, or less than 0.1% CXCR4 nucleic acid (based on the total molar amounts of the CXCR4 nucleic acid and the CCR5 nucleic acid in the mixture) is provided.

In some embodiments, a nucleic acid is a vector (optionally, a DNA vector). In some embodiments, the vector is a plasmid, cosmid, viral vector or artificial chromosome. In some embodiments, the artificial chromosome is bacterial or yeast in origin.

In some embodiments, a kit comprising nucleic acids in a container (optionally, a mixture of the nucleic acids) is provided. In some embodiments, the nucleic acids in a container are DNA vectors. In some embodiments, the nucleic acids in a container are RNA transcripts, e.g., of one of the DNA nucleic acids described herein. In some embodiments, the kit comprises containers with one or more Primer Sets and other amplification reagents.

In some embodiments, a method for amplifying (and optionally sequencing) a portion of nucleic acids (optionally, a mixture of nucleic acids) as controls is provided. In some embodiments, the nucleic acids are DNA vectors. In some embodiments, the nucleic acids are RNA transcripts, e.g., of one of the DNA nucleic acids described herein. In some embodiments, the methods amplify (and optionally sequence) a V3 region within the nucleic acids.

In some embodiments, the nucleic acid sequences of the present disclosure can be used in conjunction with a treatment for HIV infection.

### BRIEF DESCRIPTION OF THE DRAWING

FIG. 1 presents a schematic diagram illustrating an exemplary method for amplifying and sequencing the V3 region of the HIV env gene using certain primers of the present disclosure.

FIG. 2 presents a chart of CCR5 coreceptor usage (R5) as a percentage of total tropism grouped by HIV-1 subtype. The chart provides the number of R5 or non-R5 results grouped by HIV-1 subtype. The most commonly reported subtypes were A1 (74.5%), B (11.1%), and CRF_{—}01 or related viruses (10.5%). The percent R5 was 82.2% for A1, 82.4% for B, and 60% for CRF_{—}01 or related viruses. R5 usage was 80% for this patient screening population as a whole.

### DEFINITIONS

The term “active fragment”, as used herein in reference to an oligonucleotide (e.g., an oligonucleotide sequence provided herein), refers to any nucleic acid molecule which includes fewer nucleotides than the full length oligonucleotide, and retains at least one biological property of the full length oligonucleotide. For example, in some embodiments, active fragments may retain the ability to act as primers in an HIV amplification reaction. An active fragment of the present disclosure can be a nucleic acid molecule which is, for example, 10, 15, 20, 25, 30 or more nucleotides in length and can be used as a primer in an HIV amplification reaction.

The term “amplification” or “amplification reaction” is used herein to refer to any in vitro process for exponentially increasing the number of copies of a nucleotide sequence or sequences. Nucleic acid amplification results in the incorporation of nucleotides (ribonucleotides or deoxyribonucleotides) into primers to form DNA or RNA molecules that are complementary to a template nucleic acid molecule. As used herein, one amplification reaction may consist of many rounds of primer extension. For example, one PCR reaction may consist of several cycles of denaturation and extension ranging from, e.g., about 5 cycles to about 1000 cycles, or more.

The term “amplification reaction reagents”, is used herein to refer to reagents used in nucleic acid amplification reactions and may include, but are not limited to, buffers, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphosphate, deoxyguanosine triphosphate, deoxycytidine triphosphate and deoxythymidine triphosphate.

The term “gene”, as used herein, has its art understood meaning, and refers to a part of the genome specifying a macromolecular product, be it DNA for incorporation into a host genome, a functional RNA molecule or a protein, and may include regulatory sequences (e.g., promoters, enhancers, etc.) and/or intron sequences preceding (5′ non-coding sequences) and following (3′ non-coding sequences) the coding sequences.

The term “hybridization”, as used herein, refers to the formation of complexes (also called duplexes or hybrids) between nucleotide sequences which are sufficiently complementary to form complexes via Watson-Crick base pairing or non-canonical base pairing. It will be appreciated that hybridizing sequences need not have perfect complementary to provide stable hybrids. In many situations, stable hybrids will form where fewer than about 10% of the bases are mismatches. Accordingly, as used herein, the term “complementary” refers to a nucleic acid molecule that forms a stable duplex with its complement under assay conditions, generally where there is about 90% or greater homology (e.g., about 95% or greater, about 98% or greater, or about 99% or greater homology). Those skilled in the art understand how to estimate and adjust the stringency of hybridization conditions such that sequences that have at least a desired level of complementarily will stably hybridize, while those having lower complementarily will not. For examples of hybridization conditions and parameters, see, for example, Sambrook et al., “Molecular Cloning: A Laboratory Manual”, 1989, Second Edition, Cold Spring Harbor Press: Plainview, N.Y. and Ausubel, “Current Protocols in Molecular Biology”, 1994, John Wiley & Sons: Secaucus, N.J. Complementarity between two nucleic acid molecules is said to be “complete”, “total” or “perfect” if all the nucleic acid's bases are matched, and is said to be “partial” otherwise.

The terms “labeled” and “labeled with a detectable agent (or moiety)” are used herein interchangeably to specify that an entity (e.g., a target sequence) can be visualized, e.g., directly or following hybridization to another entity that comprises a detectable agent or moiety. Preferably, the detectable agent or moiety is selected such that it generates a signal which can be measured and whose intensity is related to (e.g., proportional to) the amount of the entity of interest (e.g., a target sequence). Methods for labeling nucleic acid molecules are well-known in the art. In some embodiments, labeled nucleic acids can be prepared by incorporation of, or conjugation to, a label that is directly or indirectly detectable by spectroscopic, photochemical, biochemical, immunochemical, electrical, optical, or chemical means.

The term “melting temperature” or “Tm” of a specific oligonucleotide, as used herein, refers to the specific temperature at which half of the oligonucleotide hybridizes to its target in equilibrium. Accurate prediction of the Tm of any oligonucleotide can be made based on sequence using nearest neighbor parameter calculations.

The terms “nucleic acid”, “nucleic acid molecule”, “polynucleotide” or “oligonucleotide” are used herein interchangeably. They refer to linear polymers of nucleotide monomers or analogs thereof, such as deoxyribonucleic acid (DNA) and ribonucleic acid (RNA). Unless otherwise stated, the terms encompass nucleic acid-like structures with synthetic backbones, as well as amplification products. As will be appreciated by one skilled in the art, the length of these polymers (i.e., the number of nucleotides it contains) can vary widely, often depending on their intended function or use. In some embodiments, the term “oligonucleotide” is used herein to denote a polynucleotide that comprises between about 5 and about 150 nucleotides, e.g., between about 10 and about 100 nucleotides, between about 15 and about 75 nucleotides, or between about 15 and about 50 nucleotides. Throughout the specification, whenever an oligonucleotide is represented by a sequence of letters (chosen, for example, from the four base letters: A, C, G, and T, which denote adenosine, cytidine, guanosine, and thymidine, respectively), the nucleotides are presented in the 5′→3′ order from the left to the right. In some embodiments, the sequence of an oligonucleotide of the present disclosure contains the letter M and/or letter Y and/or letter R. As used herein, the letter “M” represents a degenerative base, which can be A or C with substantially equal probability. As used herein, the letter “Y” represents a degenerative base, which can be T or C with substantially equal probability. As used herein, the letter “R” represents a degenerative base, which can be A or G with substantially equal probability. Thus, for example, in the context of the present disclosure, if an oligonucleotide contains one degenerative base M, the oligonucleotide is a substantially equimolar mixture of two subpopulations of a first oligonucleotide where the degenerative base is A and a second oligonucleotide where the degenerative base is C, the first and second oligonucleotides being otherwise identical.

The term “3′” refers to a region or position in a polynucleotide or oligonucleotide 3′ (i.e., downstream) from another region or position in the same polynucleotide or oligonucleotide. The term “5′” refers to a region or position in a polynucleotide or oligonucleotide 5′ (i.e., upstream) from another region or position in the same polynucleotide or oligonucleotide. The terms “3′ end” and “3′ terminus”, as used herein in reference to a nucleic acid molecule, refer to the end of the nucleic acid which contains a free hydroxyl group attached to the 3′ carbon of the terminal pentose sugar. The term “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar.

The term “isolated”, as used herein in reference to an oligonucleotide, means an oligonucleotide, which by virtue of its origin or manipulation, is separated from at least some of the components with which it is naturally associated or with which it is associated when initially obtained. By “isolated”, it is alternatively or additionally meant that the oligonucleotide of interest is produced or synthesized by the hand of man.

The terms “primer”, as used herein, typically refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least about 8, e.g., at least about 10, at least about 15, or about 20 to about 40 consecutive nucleotides of a target nucleic acid (i.e., will hybridize to a contiguous sequence of the target nucleic acid). In general, a primer sequence is identified as being either “complementary” (i.e., complementary to the coding or sense strand (+)), or “reverse complementary” (i.e., complementary to the anti-sense strand (−)). In some embodiments, the term “primer” may refer to an oligonucleotide that acts as a point of initiation of a template-directed synthesis using methods such as PCR (polymerase chain reaction) or LCR (ligase chain reaction) under appropriate conditions (e.g., in the presence of four different nucleotide triphosphates and a polymerization agent, such as DNA polymerase, RNA polymerase or reverse-transcriptase, DNA ligase, etc., in an appropriate buffer solution containing any necessary reagents and at suitable temperature(s)). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

The terms “forward primer” and “forward amplification primer” are used herein interchangeably, and refer to a primer that hybridizes (or anneals) to the target (template) strand. The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “primer set” is used herein to refer to two or more primers which together are capable of priming the amplification of a target nucleotide sequence (e.g., to amplify DNA or RNA encoding the HIV env gene or a portion thereof). In some embodiments, the term “primer set” refers to a pair of primers including a 5′ (upstream) primer (or forward primer) that hybridizes with the 5′-end of the nucleic acid sequence to be amplified and a 3′ (downstream) primer (or reverse primer) that hybridizes with the complement of the sequence to be amplified. Such primer set or primer pair are particularly useful in PCR amplification reactions.

As used herein, the term “nested primer set” refers to two or more primers which together are capable of priming the amplification of an amplified nucleotide sequence of interest. The primers in a “nested primer set” are sometimes referred to herein as “inner primers”. In some embodiments, one or more primers of the “nested primer set” are overlapping with primers that were used to amplify the original nucleotide sequence of interest (i.e., with “outer primers”). In some embodiments, the “nested primer set” is non-overlapping with primers that were used to amplify the original nucleotide sequence of interest. In some embodiments, the term “nested primer set” refers to a pair of primers including a 5′ (upstream) primer (or forward primer) that hybridizes with or towards the 5′-end of the amplified nucleic acid sequence of interest and a 3′ (downstream) primer (or reverse primer) that hybridizes with or towards the 5′-end of the complement of the amplified nucleic acid sequence of interest.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, obtained cells are or include microbial cells of an individual's microbiome. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., plasma, blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “secondary sample” or “processed sample” may comprise, for example nucleic acids or proteins extracted from a “primary sample” or obtained by subjecting a “primary sample” to techniques such as amplification or reverse transcription of mRNA, isolation and/or purification of certain components, etc.

The term “target nucleic acid sequence” or “nucleic acid of interest” is used herein to refer to any series of contiguous nucleotides in a template nucleic acid molecule (such as DNA, cDNA or RNA) to be amplified. One specific target nucleic acid sequence is a segment, region, or fragment of a nucleic acid molecule that hybridizes to at least one inner primer during a nested PCR reaction.

The term “tropism” is used herein to refer to the affinity of a viral particle (such as HIV) for particular cell and receptor types (such as CCR5 or CXCR4). “Tropic variant” is used herein to refer to HIV genomic sequence variations associated with a tropism.

### DETAILED DESCRIPTION OF CERTAIN EMBODIMENTS

HIV is a member of the lentivirus family of retroviruses. Retroviruses are small enveloped viruses that contain a single-stranded RNA genome and replicate via a DNA intermediate produced by a virally-encoded reverse transcriptase, an RNA-dependent DNA polymerase. HIV can be divided into two major types, HIV type 1 (HIV-1) and HIV type 2 (HIV-2) that account for the vast majority of viral isolates.

The HIV viral particle comprises a viral core, composed in part of capsid proteins, together with the viral RNA genome and those enzymes required for early replicative events. Myristylated gag protein forms an outer shell around the viral core, which is, in turn, surrounded by a lipid membrane envelope derived from the infected cell membrane. The envelope further comprises viral glycoproteins encoded by the HIV env gene.

The HIV envelope protein has been extensively described, and the amino acid and RNA sequences encoding HIV envelope from a number of HIV strains are known (Myers, G. et al., 1992. “Human Retroviruses and AIDS. A compilation and analysis of nucleic acid and amino acid sequences.” Los Alamos National Laboratory, Los Alamos, N. Mex.). The HIV envelope protein is a glycoprotein of about 160 kd (gp160) which is anchored in the membrane bilayer at its carboxyl terminal region. The N-terminal segment, gp120, protrudes into the aqueous environment surrounding the virion and the C-terminal segment, gp41, spans the membrane. Via a host-cell mediated process, gp160 is cleaved to form gp120 and the integral membrane protein gp41. As there is no covalent attachment between gp120 and gp41, free gp120 is released from the surface of virions and infected cells.

The gp120 molecule consists of a polypeptide core of 60,000 daltons which is extensively modified by N-linked glycosylation to increase the apparent molecular weight of the molecule to 120,000 daltons. The amino acid sequence of gp120 contains five relatively conserved domains interspersed with five hypervariable domains. The positions of the 18 cysteine residues in the gp120 primary sequence, and the positions of 13 of the approximately 24 N-linked glycosylation sites in the gp120 sequence are common to all gp120 sequences. The hypervariable domains contain extensive amino acid substitutions, insertions and deletions. Sequence variations in these domains result in up to 30% overall sequence variability between gp120 molecules from the various viral isolates. Despite this variation, all gp120 sequences preserve the virus's ability to bind to the viral receptor CD4 and to interact with gp41 to induce fusion of the viral and host cell membranes.

Because the CD4 receptor acts as the cellular receptor for the HIV-1 virus, HIV-1 is targeted to CD4⁺ cells (Maddon et al., “The T4 gene encodes the AIDS virus receptor and is expressed in the immune system and the brain,” 1986, Cell 47:333-348). Viral entry into cells is dependent upon gp120 binding the cellular CD4 receptor molecules (McDougal, J. S., et al., 1986, “Binding of HTLV-III/LAV to T4+ T cells by a complex of the 110K viral protein and the T4 molecule,” Science 231:382-385) while gp41 anchors the envelope glycoprotein complex in the viral membrane. While these virus-cell interactions are necessary for infection, additional virus-cell interactions are also required.

HIV-1 cell entry via the CD4 receptor is facilitated by a co-receptor molecule. Tropism is co-receptor specificity of a given viral particle. The majority of HIV-1 strains utilize the chemokine receptors CCR5, CXCR4 or both. Most newly infected individuals appear to have predominantly CCR5 tropic virus. Additional examples of HIV tropism strains and their relationship to disease progression are described in Poveda et al. (“HIV tropism: diagnostic tools and implications for disease progression and treatment with entry inhibitors,” AIDS 2006, 20:1359-1367), Jensen et al. (“Predicting HIV-1 coreceptor usage with sequence analysis,” AIDS Rev 2003; 5:104-112), Jensen et al. (“A reliable phenotype predictor for human immunodeficiency virus type 1 subtype C based on envelope V3 sequences,” Journal of Virology, May 2006, p. 4698-4704), Jensen et al. (“Improved coreceptor usage prediction and genotypic monitoring of R5-to-X4 transition by motif analysis of human immunodeficiency virus type 1 env V3 loop sequences,” Journal of Virology, December 2003, p. 13376-13388), and Nelson et al. (“Evolutionary variants of the human immunodeficiency virus type 1 V3 region characterized by using a heteroduplex tracking assay,” Journal of Virology, November 1997, p. 8750-8758), each of which is hereby incorporated by reference herein in its entirety.

The CCR5 and CXCR4 co-receptors are attractive targets for drug development since they are members of the G protein-coupled receptor superfamily, a group of proteins targeted by several commonly used and well-tolerated drugs, such as desloradine, ranitidine and tegaserod. CCR5 is of particular interest since a natural polymorphism exists in humans (CCR5-Δ32) that leads to reduced or absent cell surface expression of CCR5 in heterozygotic or homozygotic genotypes, respectively. Individuals homozygotic for CCR5-Δ32 appear to benefit from a natural resistance to HIV infection, while heterozygotic CCR5-Δ32 is associated with reduced disease progression (Marmor, M. et al. “Homozygous and heterozygous CCR5-Delta32 genotypes are associated with resistance to HIV infection,” J Acquir Immune Defic Syndr. 2001 August 15; 27(5):472-81).

Maraviroc (also known as SELZENTRY, which is marketed by Pfizer Inc.) is a small molecule CCR5 agonist. Current FDA recommendations state that each patient's HIV population be tested for tropism before Maraviroc is prescribed. Clonal analysis of HIV quasispecies in patients that failed treatment during Maraviroc clinical trials revealed small amounts of CXCR4 tropic viruses present before treatment initiation were given a selective advantage over the majority CCR5 strains leading to more efficient outgrowth of CXCR4 tropic virus under drug treatment and accelerated treatment failure (see, for example, Kramer, V. G., et al. “Maraviroc and Other HIV-1 Entry Inhibitors Exhibit a Class-Specific Redistribution Effect that Results in Increased Extracellular Viral Load,” Antimicrob Agents Chemother. 2012 May 21. [Epub ahead of print]).

Viral tropism is determined by exposed amino acid sequences in the gp120 surface envelope protein. In particular, the V3 (third variable) region has been implicated in co-receptor usage selection. As the name implies, the approximately 35 amino acid long sequence is highly variable, but there are common features distinguishing CCR5 and CXCR4 tropic viruses located within this sequence. A number of tropism prediction algorithms have been developed based directly on V3 sequences. For example, several position specific scoring matrix (PSSM) algorithms that directly correlate amino acid residues in the V3 to tropism phenotypes (as well as these can be determined) have been published. (See, for example, McDonald, R. A., “Relationship between V3 genotype, biologic phenotype, tropism, and coreceptor use for primary isolates of human immunodeficiency virus type 1,” J Hum Virol. 2001 July-August; 4(4):179-87).

A number of assays for determining the tropism of an HIV population are known in the art. These assays involve either determining the binding to receptors displayed on cell surfaces or inferring tropism from genetic information. For example, U.S. Pat. No. 7,294,458 describes an assay that involves transforming cells with an HIV envelope gene cloned from an infected patient, selectively fusing the cells with an indicator cell line that expresses an HIV envelope-compatible co-receptor and then assaying for fusion. Cell surface envelope protein variants selectively interact with either CCR5 or CXCR4 co-receptors. Fusion occurs only when an envelope protein interacts with a compatible co-receptor present on the surface of indicator cells. Cells expressing a particular envelope gene will fuse either CCR5 or CXCR4 indicator cells depending on the patient's envelope gene specificity. Fusion with either CCR5 or CXCR4 indicator cells indicates the type of co-receptor usage.

As illustrated by the assay described in U.S. Pat. No. 7,294,458, the HIV tropism assays known in the art are time consuming and are expensive. Therefore, there is a need in the art for assays for determining the tropism of an HIV population that can be performed rapidly and are less expensive to perform than assays currently known in the art.

### I—Oligonucleotide Sequences and Amplification Primer Sets

### Oligonucleotide Sequences

Previous research has identified HIV viral tropism with varying susceptibility to treatment with CCR5 agonists. In once aspect, the present disclosure provides oligonucleotide sequences for determining HIV coreceptor tropism (CCR5 or CXCR4) by amplifying the V3 region of the HIV env genomic sequence or a portion thereof.

DNA sequencing assays for HIV have limited performance due to the high variability of the HIV genome. The main mode of failure is due to poor performance of oligonucleotides meant to initiate reverse transcription followed by PCR based amplification of the viral RNA. There is a need to improve the sensitivity performance beyond what is currently possible.

For the particular region targeted, the V3 region of the HIV env gene, the design that was developed is likely to exhibit unique performance compared to other possible designs for the purpose of in vitro reverse transcription and PCR amplification of the V3 region of the HIV env gene or a portion thereof. In some embodiments, this amplicon can be further analyzed by direct DNA sequencing, or nested PCR followed by DNA sequencing, to determine mutations associated with coreceptor tropism in the V3 region of HIV. In general any sequencing method can be used for this purpose (e.g., Sanger Sequencing, Next Generation Sequencing and so-called Third Generation Sequencing). In some embodiments, it can be cloned and expressed in model organisms for HIV disease studies and pharmaceutical development programs. In some embodiments, the amplified patient-derived product may also be cloned for in vitro phenotyping experiments with pseudotyped virus.

The sequences of certain oligonucleotides of the present disclosure are set forth in Tables 1 (SEQ ID NOS: 1-35).

TABLE 1

| SEQ ID NO: | Sequence Name | Sequence (5′ → 3′) | Strand |
|---|---|---|---|
| 1 | EMF1 | AGAGAAAGAGCAGAAGACAGTGGM | (+) |
| 2 | EMF1-1 | AGAGAAAGAGCAGAAGACAGTGGC | (+) |
| 3 | EMF1-2 | AGAGAAAGAGCAGAAGACAGTGGA | (+) |
| 4 | EMR1 | CCTTGTAAGTCATTGGTCTTAAAGGTACY | (−) |
| 5 | EMR1-1 | CCTTGTAAGTCATTGGTCTTAAAGGTACC | (−) |
| 6 | EMR1-2 | CCTTGTAAGTCATTGGTCTTAAAGGTACT | (−) |
| 7 | 6957F | GTACAATGTACACATGGAAT | (+) |
| 8 | 7371 | AAAATTCTCCTCTACARTTA | (−) |
| 9 | 7371-2R | AAAATTCTCCTCTACAATTA | (−) |
| 10 | 7371-3R | AAAATTCTCCTCTACAGTTA | (−) |
| 11 | 7371-4R-M1 | AAAATTCTCCTCCACAATT | (−) |
| 12 | Italian_RT_F | CAGCACAGTACARTGTACACA | (+) |
| 13 | Italian_RT_R | CTTCTCCAATTGTCYYTCA | (−) |
| 14 | Italian_Seq_F | CTGTTAAATGGYAGYCTAGC | (+) |
| 15 | Italian_Seq_R | CAATTTCTRGGTCYCCTC | (−) |
| 16 | Italian_RT_1F | CAGCACAGTACAATGTACACA | (+) |
| 17 | Italian_RT_2F | CAGCACAGTACAGTGTACACA | (+) |
| 18 | Italian_RT_1R | CTTCTCCAATTGTCCCTCA | (−) |
| 19 | Italian_RT_2R | CTTCTCCAATTGTCCTTCA | (−) |
| 20 | Italian_RT_3R | CTTCTCCAATTGTCTCTCA | (−) |
| 21 | Italian_RT_4R | CTTCTCCAATTGTCTTTCA | (−) |
| 22 | Italian_Seq_1F | CTGTTAAATGGCAGTCTAGC | (+) |
| 23 | Italian_Seq_2F | CTGTTAAATGGCAGCCTAGC | (+) |
| 24 | Italian_Seq_3F | CTGTTAAATGGTAGTCTAGC | (+) |
| 25 | Italian_Seq_4F | CTGTTAAATGGTAGCCTAGC | (+) |
| 26 | Italian_Seq_1R | CAATTTCTGGGTCCCCTC | (−) |
| 27 | Italian_Seq_2R | CAATTTCTGGGTCTCCTC | (−) |
| 28 | Italian_Seq_3R | CAATTTCTAGGTCCCCTC | (−) |
| 29 | Italian_Seq_4R | CAATTTCTAGGTCTCCTC | (−) |
| 30 | EMF1_3 | AGAGAAAGAGCAGAAGACAGTGG | (+) |
| 31 | EMR1_3 | CCTTGTAAGTCATTGGTCTTAAAGGTAC | (−) |
| 32 | 7371R | AAAATTCCCCTCCACAATTA | (+) |
| 33 | 7371R-M1 | AAAATTCCCCTCCACAATT | (−) |
| 34 | Ml3F BP | TTCTGGCGTACCGTTCCTGTC | (+) |
| 35 | M13R BP | GTTTTCCCAGTCACGACGTTGTA | (−) |

### Amplification Primer Sets

Oligonucleotides of the present disclosure may be conveniently provided as primer sets that can be used to amplify an V3 region of the HIV env gene, e.g., to determine which polymorphic variant(s) is/are present among some or all of the possible polymorphic variants that may exist at a particular polymorphic site. Multiple sets of primers, capable of detecting polymorphic variants at a plurality of polymorphic sites are provided.

Examples of primer sets/pairs comprising a forward amplification primer and a reverse amplification primer include:
Primer Set 1, which comprises a forward primer comprising SEQ ID NO: 1, or any active fragment thereof, and a reverse primer comprising SEQ ID NO: 4, or any active fragment thereof,
Primer Set 2, which comprises a forward primer comprising SEQ ID NO: 7, or any active fragment thereof, and a reverse primer comprising SEQ ID NO: 8 or SEQ ID NO: 11 or any active fragments or combinations thereof,
Primer Set 3, which comprises a forward primer comprising SEQ ID NO: 12, or any active fragment thereof, and a reverse primer comprising SEQ ID NO: 13, or any active fragment thereof,
Primer Set 4, which comprises a forward primer comprising SEQ ID NO: 14, or any active fragment thereof, and a reverse primer comprising SEQ ID NO: 15, or any active fragment thereof,
Primer Set 5, which comprises a forward primer comprising SEQ ID NO: 2 or SEQ ID NO: 3, or any active fragments or combinations thereof, and a reverse primer comprising SEQ ID NO: 5 or SEQ ID NO: 6 or any active fragments or combinations thereof, and
Primer Set 6, which comprises a forward primer comprising SEQ ID NO: 7, or any active fragment thereof, and a reverse primer comprising SEQ ID NO: 9, SEQ ID NO: 10, or SEQ ID NO: 11 or any active fragments or combinations thereof,
Primer Set 7, which comprises a forward primer comprising SEQ ID NO: 16 or SEQ ID NO: 17, or any active fragments or combinations thereof, and a reverse primer comprising SEQ ID NO: 18, SEQ ID NO: 19, SEQ ID NO: 20, or SEQ ID NO: 21, or any active fragments or combinations thereof,
Primer Set 8, which comprises a forward primer comprising SEQ ID NO: 22, SEQ ID NO: 23, SEQ ID NO: 24, or SEQ ID NO: 25, or any active fragments or combinations thereof, and a reverse primer comprising SEQ ID NO: 26, SEQ ID NO: 27, SEQ ID NO: 28, or SEQ ID NO: 29, or any active fragments or combinations thereof,
Primer Set 9, which comprises a forward primer comprising SEQ ID NO: 2, SEQ ID NO: 3, or SEQ ID NO: 30, or any active fragments or combinations thereof, and a reverse primer comprising SEQ ID NO: 5, SEQ ID NO: 6, or SEQ ID NO: 31, or any active fragments or combinations thereof, and
Primer Set 10, which comprises a forward primer comprising SEQ ID NO: 7, or any active fragment thereof, and a reverse primer comprising SEQ ID NO: 9, SEQ ID NO: 10, SEQ ID NO: 11, SEQ ID NO: 32, or SEQ ID NO: 33, or any active fragments or combinations thereof.

### Oligonucleotide Preparation

Oligonucleotides of the present disclosure may be prepared by any of a variety of methods (see, e.g., Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, Innis (Ed.), Academic Press: New York, N.Y.; Tijssen “Hybridization with Nucleic Acid Probes—Laboratory Techniques in Biochemistry and Molecular Biology (Parts I and II)”, 1993, Elsevier Science; “PCR Strategies”, 1995, Innis (Ed.), Academic Press: New York, N.Y.; and “Short Protocols in Molecular Biology”, 2002, Ausubel (Ed.), 5^{th} Ed., John Wiley & Sons: Secaucus, N.J.).

In some embodiments, oligonucleotides may be prepared by chemical techniques well-known in the art, including, e.g., chemical synthesis and polymerization based on a template as described, e.g., in Narang et al., Meth. Enzymol. 68:90-98 (1979); Brown et al., Meth. Enzymol. 68: 109-151 (1979); Belousov et al., Nucleic Acids Res. 25:3440-3444 (1997); Guschin et al., Anal. Biochem. 250:203-211 (1997); Blommers et al., Biochemistry 33:7886-7896 (1994); Frenkel et al., Free Radic. Biol. Med. 19:373-380 (1995); and U.S. Pat. No. 4,458,066.

In some embodiments, oligonucleotides may be prepared using an automated, solid-phase procedure based on the phosphoramidite approach. In such methods, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of oligonucleotides, where necessary or desirable, may be carried out by any of a variety of methods well-known in the art. For example, purification of oligonucleotides is typically performed either by native acrylamide gel electrophoresis, by anion-exchange HPLC, e.g., see Pearson and Regnier, J. Chrom. 255:137-149 (1983) or by reverse phase HPLC, e.g., see McFarland and Borer, Nucleic Acids Res. 7:1067-1080 (1979).

The sequence of oligonucleotides can be verified using any suitable sequencing method including, but not limited to, chemical degradation, e.g., see Maxam and Gilbert, Methods of Enzymology, 65:499-560 (1980), matrix-assisted laser desorption ionization time-of-flight (MALDI-TOF) mass spectrometry, e.g., see Pieles et al., Nucleic Acids Res. 21:3191-3196 (1993), mass spectrometry following a combination of alkaline phosphatase and exonuclease digestions, e.g., see Wu and Aboleneen, Anal. Biochem. 290:347-352 (2001).

The present disclosure encompasses modified versions of these oligonucleotides that perform as equivalents of these oligonucleotides in accordance with the methods of the present disclosure. These modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.), or charged linkages (e.g., phosphorothioates, phosphorodithioates, etc.). Modified oligonucleotide may also be derivatized by formation of a methyl or ethyl phosphotriester or an alkyl phosphoramidate linkage. Furthermore, the oligonucleotides of the present disclosure may also be modified with a label.

### Labeling of Oligonucleotides

In some embodiments, the primers are labeled with a detectable agent or moiety before being used in amplification/detection assays. The role of a detectable agent is to allow visualization and detection of amplified target sequences. Preferably, the detectable agent is selected such that it generates a signal which can be measured and whose intensity is related (e.g., proportional) to the amount of amplification products in the sample being analyzed.

The association between the oligonucleotide and the detectable agent can be covalent or non-covalent. Labeled detection primers can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules, e.g., see Mansfield et al., Mol. Cell Probes 9:145-156 (1995).

Various methods for labeling nucleic acid molecules are known in the art. For a review of labeling protocols, label detection techniques, and recent developments in the field, see, for example, Kricka, Ann. Clin. Biochem. 39:114-129 (2002); van Gijlswijk et al., Expert Rev. Mol. Diagn. 1:81-91 (2001); and Joos et al., J. Biotechnol. 35:135-153 (1994). Standard nucleic acid labeling methods include: incorporation of radioactive agents, direct attachments of fluorescent dyes (Smith et al., Nucl. Acids Res. 13:2399-2412 (1985)) or of enzymes (Connoly and Rider, Nucl. Acids. Res. 13:4485-4502 (1985)); chemical modifications of nucleic acid molecules making them detectable immunochemically or by other affinity reactions, e.g., see Broker et al., Nucl. Acids Res. 5:363-384 (1978); Bayer et al., Methods of Biochem. Analysis 26:1-45 (1980); Langer et al., Proc. Natl. Acad. Sci. USA 78:6633-6637 (1981); Richardson et al., Nucl. Acids Res. 11:6167-6184 (1983); Brigati et al., Virol. 126:32-50 (1983); Tchen et al., Proc. Natl. Acad. Sci. USA 81:3466-3470 (1984); Landegent et al., Exp. Cell Res. 15:61-72 (1984); and Hopman et al., Exp. Cell Res. 169:357-368 (1987); and enzyme-mediated labeling methods, such as random priming, nick translation, PCR and tailing with terminal transferase. For a review on enzymatic labeling, see, e.g., Temsamani and Agrawal, Mol. Biotechnol. 5:223-232 (1996). More recently developed nucleic acid labeling systems include, but are not limited to: ULS (Universal Linkage System), which is based on the reaction of monoreactive cisplatin derivatives with the N7 position of guanine moieties in DNA (Heetebrij et al., Cytogenet. Cell. Genet. 87:47-52 (1999)), psoralen-biotin, which intercalates into nucleic acids and upon UV irradiation becomes covalently bonded to the nucleotide bases (Levenson et al., Methods Enzymol. 184:577-583 (1990); and Pfannschmidt et al., Nucleic Acids Res. 24:1702-1709 (1996)), photoreactive azido derivatives (Neves et al., Bioconjugate Chem. 11:51-55 (2000)), and DNA alkylating agents (Sebestyen et al., Nat. Biotechnol. 16: 568-576 (1998)).

It will be appreciated that any of a wide variety of detectable agents can be used in the practice of the present disclosure. Suitable detectable agents include, but are not limited to, various ligands, radionuclides (such as, for example, ³²P, ³⁵S, ³H, ¹⁴C, ¹²⁵I, ¹³¹ I, and the like); fluorescent dyes; chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, e.g., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

A “tail” of normal or modified nucleotides can also be added to tag an oligonucleotide for detectability purposes. In some embodiments, an M13 tag sequence (SEQ ID NO: 34 or 35) may be added.

### Nucleic Acid Controls

In some embodiments, the present disclosure provides one or more nucleic acids that can be used as controls in kits or methods of the present disclosure. In some embodiments, the nucleic acids are DNA based (e.g., DNA vectors). In some embodiments, the nucleic acids are RNA based (e.g., RNA transcripts). In some embodiments, these nucleic acids comprise a sequence that encodes all or a portion of an HIV Env protein or variant thereof (e.g., a sequence that encodes a protein that is at least 80%, 85%, 90%, 95% or 99% homologous to an HIV Env protein). In some embodiments, the HIV Env protein is from (or derived from) the US1 or US2 clone. In some embodiments, these nucleic acids comprise hybridization sites for at least one of the aforementioned Primer Sets (e.g., one or more of Primer Sets 1-10). In some embodiments, these hybridization sites are perfect complements for the primers in the one or more Primer Sets. In some embodiments, these nucleic acids comprise hybridization sites for each of the primers in Primer Sets 1-10.

In some embodiments, the nucleic acids encodes a portion of an HIV Env protein that includes the V3 region. In some embodiments, the sequence that encodes all or a portion of an HIV Env protein or variant thereof comprises a V3 region sequence that is at least 80%, 85%, 90%, 95% or 99% homologous to the following sequence (or an RNA equivalent):

(SEQ ID NO.: 42)
TGTACAAGACCCAACAACAATACAAGAAAAAGTATACATATAGGACCAG
GGAGAGCATTTTATGCAACAGGAGAAATAATAGGAGATATAAGACAAGC
ACATTGT.

In some embodiments, the sequence that encodes all or a portion of an HIV Env protein or variant thereof comprises a V3 region sequence that is at least 80%, 85%, 90%, 95% or 99% homologous to the following sequence (or an RNA equivalent):

(SEQ ID NO.: 43)
TGTACAAGACCCAACAACAATACAAGAAAAAGTATACGTATAGGACCAG
GGAGAGCATTTTATGCAACAGGAAAAATAATAGGAGATATAAGACAAGC
ACATTGT.

In some embodiments, the sequence that encodes all or a portion of an HIV Env protein or variant thereof comprises a V3 region sequence that is at least 80%, 85%, 90%, 95% or 99% homologous to the following sequence (or an RNA equivalent):

(SEQ ID NO.: 44)
TGTACAAGACCCAGCAACAATACAAGAAAAAGTATACATATAGGACCAG
GGAGAGCATTTTATACAACAGGAAATATAATAGGAGATATAAGACAAGC
ACATTGT.

In some embodiments, the sequence that encodes all or a portion of an HIV Env protein or variant thereof comprises a V3 region sequence that is at least 80%, 85%, 90%, 95% or 99% homologous to the following sequence (or an RNA equivalent):

(SEQ ID NO.: 45)
TGCATAAGACCCAACAACAATACAAGAAAAAGTATACATATAGGACCAG
GGAGAGCAATTTATGCAACAGGAGGCATAATAGGAGATATAAGACGAGC
ATATTGT.

In some embodiments, the sequence that encodes all or a portion of an HIV Env protein or variant thereof comprises a V3 region sequence that is at least 80%, 85%, 90%, 95% or 99% homologous to the following sequence (or an RNA equivalent):

(SEQ ID NO.: 46)
TGTACAAGACCCAACAACAATACAAGAAAAAGTATACATATAGGACCAG
GGAGAGCATTTTATGCAACAGGAGAAATAATAGGAGATATAAGACAAGC
ACATTGT.

In some embodiments, the sequence that encodes all or a portion of an HIV Env protein or variant thereof comprises a V3 region sequence that is at least 80%, 85%, 90%, 95% or 99% homologous to the following sequence (or an RNA equivalent):

(SEQ ID NO.: 47)
TGTACAAGACCCAACAACAATACAAGAAAAAGTATACGTATAGGACCAG
GGAGAGCATTTTATGCAACAGGAAAAATAATAGGAGATATAAGACAAGC
ACATTGT.

In some embodiments, the present disclosure provides nucleic acids that produce an amplicon that includes the V3 region of an HIV Env protein when amplified using at least one of the aforementioned Primer Sets (e.g., one or more of Primer Sets 1-10) (optionally after a reverse transcription step when the nucleic acid is an RNA transcript).

In some embodiments, the present disclosure provides a nucleic acid comprising a sequence that encodes all or a portion of a CCR5 coreceptor variant HIV Env protein. In other embodiments, the nucleic acid sequence encodes all or a portion of a CXCR4 coreceptor variant HIV Env protein. In some embodiments, the nucleic acid sequence encodes a truncated CCR5 coreceptor variant HIV Env protein. In some embodiments, the nucleic acid sequence encodes a truncated CXCR4 coreceptor variant HIV Env protein.

In some embodiments, the present disclosure provides a nucleic acid that comprises a sequence encoding an HIV Env protein or variant thereof where the sequence is at least 80%, 85%, 90%, 95% or 99% homologous with a sequence selected from the group consisting of SEQ ID NOs.: 36, 37, 38, 39, 40 and 41 (or an RNA equivalent). In certain embodiments, the sequence also comprises a sequence selected from the group consisting of SEQ ID NOs.: 42, 43, 44, 45, 46 and 47 (or an RNA equivalent). In some embodiments, the present disclosure provides a nucleic acid that comprises a sequence selected from the group consisting of SEQ ID NOs.: 36, 37, 38, 39, 40 and 41 (or an RNA equivalent).

In some embodiments, the present disclosure provides a nucleic acid that comprises a sequence encoding an HIV Env protein or variant thereof where the sequence is at least 80%, 85%, 90%, 95% or 99% homologous with the sequence defined by nucleotides 76 through 2916 of SEQ ID NO.: 36 or 38 (or an RNA equivalent). In certain embodiments, the sequence also comprises SEQ ID NO.: 42 or 44. In some embodiments, the present disclosure provides a nucleic acid that comprises the sequence defined by nucleotides 76 through 2916 of SEQ ID NO.: 36 or 38 (or an RNA equivalent).

In some embodiments, the present disclosure provides a nucleic acid that comprises a sequence encoding an HIV Env protein or variant thereof where the sequence is at least 80%, 85%, 90%, 95% or 99% homologous with the sequence defined by nucleotides 76 through 2916 of SEQ ID NO.: 37 or 39 (or an RNA equivalent). In certain embodiments, the sequence also comprises SEQ ID NO.: 43 or 45 (or an RNA equivalent). In some embodiments, the present disclosure provides a nucleic acid that comprises the sequence defined by nucleotides 76 through 2916 of SEQ ID NO.: 37 or 39 (or an RNA equivalent).

In some embodiments, the disclosure provides mixtures of two or more of the aforementioned nucleic acids (e.g., two or more vectors). In some embodiments, the mixtures comprise a first nucleic acid (e.g., a first vector) that encodes all or a portion of a CCR5 coreceptor variant HIV Env protein (hereinafter a “CCR5 nucleic acid”) and a second nucleic acid (e.g., a second vector) that encodes all or a portion of a CXCR4 coreceptor variant HIV Env protein (hereinafter a “CXCR4 nucleic acid”). It is to be understood that any of the aforementioned CCR5 or CXCR4 nucleic acids may be used in a mixture (e.g., a nucleic acid sequence that comprises SEQ ID NOs.: 36, 37, 38, 39, 40 or 41 (or an RNA equivalent) or a portion or homolog thereof).

In some embodiments, a mixture may comprise a first nucleic acid (e.g., a first vector) that comprises the sequence of SEQ ID NO.: 36 (or a sequence that is at least 80%, 85%, 90%, 95% or 99% homologous to the sequence of SEQ ID NO.: 36) (or an RNA equivalent) and a second nucleic acid (e.g., a second vector) that comprises the sequence of SEQ ID NO.: 37 (or a sequence that is at least 80%, 85%, 90%, 95% or 99% homologous to the sequence of SEQ ID NO.: 37) (or an RNA equivalent).

In some embodiments, a mixture may comprise a first nucleic acid (e.g., a first vector) that comprises the sequence defined by nucleotides 76 through 2916 of SEQ ID NO.: 36 (or a sequence that is at least 80%, 85%, 90%, 95% or 99% homologous with the sequence defined by nucleotides 76 through 2916 of SEQ ID NO.: 36) (or an RNA equivalent) and a second nucleic acid (e.g., a second vector) that comprises the sequence defined by nucleotides 76 through 2916 of SEQ ID NO.: 37 (or a sequence that is at least 80%, 85%, 90%, 95% or 99% homologous with the sequence defined by nucleotides 76 through 2916 of SEQ ID NO.: 37) (or an RNA equivalent).

In some embodiments, a mixture may comprise a first nucleic acid (e.g., a first vector) that comprises the sequence of SEQ ID NO.: 38 (or a sequence that is at least 80%, 85%, 90%, 95% or 99% homologous to the sequence of SEQ ID NO.: 38) (or an RNA equivalent) and a second nucleic acid (e.g., a second vector) that comprises the sequence of SEQ ID NO.: 39 (or a sequence that is at least 80%, 85%, 90%, 95% or 99% homologous to the sequence of SEQ ID NO.: 39) (or an RNA equivalent).

In some embodiments, a mixture may comprise a first nucleic acid (e.g., a first vector) that comprises the sequence defined by nucleotides 76 through 2916 of SEQ ID NO.: 38 (or a sequence that is at least 80%, 85%, 90%, 95% or 99% homologous with the sequence defined by nucleotides 76 through 2916 of SEQ ID NO.: 38) (or an RNA equivalent) and a second nucleic acid (e.g., a second vector) that comprises the sequence defined by nucleotides 76 through 2916 of SEQ ID NO.: 39 (or a sequence that is at least 80%, 85%, 90%, 95% or 99% homologous with the sequence defined by nucleotides 76 through 2916 of SEQ ID NO.: 39) (or an RNA equivalent).

In some embodiments, a mixture may comprise a first nucleic acid (e.g., a first vector) that comprises the sequence of SEQ ID NO.: 40 (or a sequence that is at least 80%, 85%, 90%, 95% or 99% homologous to the sequence of SEQ ID NO.: 40) (or an RNA equivalent) and a second nucleic acid (e.g., a second vector) that comprises the sequence of SEQ ID NO.: 41 (or a sequence that is at least 80%, 85%, 90%, 95% or 99% homologous to the sequence of SEQ ID NO.: 41) (or an RNA equivalent).

In some embodiments, the mixture comprises one of the aforementioned CCR5 nucleic acids and one of the aforementioned CXCR4 nucleic acids where the molar concentration of the CCR5 nucleic acid is greater than the molar concentration of the CXCR4 nucleic acid (e.g., at least 2, 3, 5, 10, 25, 50 or at least 100 times greater). In some embodiments, the molar ratio of CXCR4 nucleic acid to CCR5 nucleic acid is less than 1:4, 1:5, 1:6, 1:8, 1:10, 1:20, 1:50 or less than 1:100. In some embodiments, the mixture comprises less than 15, 10, 8, 6, 4, 2, 1, or less than 0.1% CXCR4 nucleic acid (based on the total molar amounts of the CXCR4 nucleic acid and the CCR5 nucleic acid in the mixture).

In some embodiments, the nucleic acids are vectors (e.g., DNA vectors). In some embodiments, the vector is a plasmid, cosmid, viral vector or artificial chromosome. In some embodiments, the artificial chromosome is bacterial or yeast in origin.

In some embodiments, the nucleic acids are RNA transcripts. In some embodiments, the RNA transcripts are generated from a vector where the corresponding DNA sequence is under the control of a promoter, e.g., a T7 promoter. In some embodiments, RNA transcripts can be mixed at one of the aforementioned molar ratios of CCR5 nucleic acid and CXCR4 nucleic acid to provide a control mixture that can be used to assess or confirm detection sensitivity of CXCR4. In particular RNA equivalents of DNA sequences SEQ ID NOs.: 36-47 and portions or homologs thereof can be used in methods of the present disclosure.

In some embodiments, the present disclosure provides kits that comprise one or more of the aforementioned nucleic acids in containers (e.g., a mixture of the aforementioned nucleic acids in a single container). In some embodiments, the kits comprise containers with other reagents described herein (e.g., one or more Primer Sets and other amplification reagents).

In some embodiments, the present disclosure provides methods that involve amplifying (and optionally sequencing) a portion of these nucleic acids (optionally in the context of a mixture) as controls. In some embodiments, the methods amplify (and optionally sequence) a V3 region within these nucleic acids.

(CCR5_v3_B consensus in NC1802_ENV region):
SEQ ID NO 36
GTGTGGTCCATAGTAATCATAGAATATAGGAAAATATTAAGACAAAGAAAAATAGACAGGTTA
ATTGATAGACTAAGAGAAAGAGCAGAAGACAGTGGCAATGAGAGTGAAGGAGAAATATCAGCA
CTTGTGGAGATGGGGGTGGAGATGGGGCACCATGCTCCTTGGGATGTTGATGATCTGTAGTGC
TACAGAAAAATTGTGGGTCACAGTCTATTATGGGGTACCTGTGTGGAAGGAAGCAACCACCAC
TCTATTTTGTGCATCAGATGCTAAAGCATATGATACAGAGGTACATAATGTTTGGGCCACACA
TGCCTGTGTACCCACAGACCCCAACCCACAAGAAGTAGTATTGGTAAATGTGACAGAAAATTT
TAACATGTGGAAAAATGACATGGTAGAACAGATGCATGAGGATATAATCAGTTTATGGGATCA
AAGCCTAAAGCCATGTGTAAAATTAACCCCACTCTGTGTTAGTTTAAAGTGCACTGATTTGAA
GAATGATACTAATACCAATAGTAGTAGCGGGAGAATGATAATGGAGAAAGGAGAGATAAAAAA
CTGCTCTTTCAATATCAGCACAAGCATAAGAGGTAAGGTGCAGAAAGAATATGCATTTTTTTA
TAAACTTGATATAATACCAATAGATAATGATACTACCAGCTATAAGTTGACAAGTTGTAACAC
CTCAGTCATTACACAGGCCTGTCCAAAGGTATCCTTTGAGCCAATTCCCATACATTATTGTGC
CCCGGCTGGTTTTGCGATTCTAAAATGTAATAATAAGACGTTCAATGGAACAGGACCATGTAC
AAATGTCAGCACAGTACAATGTACACATGGAATTAGGCCAGTAGTATCAACTCAACTGCTGTT
AAATGGCAGTCTAGCAGAAGAAGAGGTAGTAATTAGATCTGTCAATTTCACGGACAATGCTAA
AACCATAATAGTACAGCTGAACACATCTGTAGAAATTAATTGTACAAGACCCAACAACAATAC
AAGAAAAAGTATACATATAGGACCAGGGAGAGCATTTTATGCAACAGGAGAAATAATAGGAGA
TATAAGACAAGCACATTGTAACATTAGTAGAGCAAAATGGAATAACACTTTAAAACAGATAGC
TAGCAAATTAAGAGAACAATTTGGAAATAATAAAACAATAATCTTTAAGCAATCCTCAGGAGG
GGACCCAGAAATTGTAACGCACAGTTTTAATTGTGGAGGGGAATTTTTCTACTGTAATTCAAC
ACAACTGTTTAATAGTACTTGGTTTAATAGTACTTGGAGTACTGAAGGGTCAAATAACACTGA
AGGAAGTGACACAATCACCCTCCCATGCAGAATAAAACAAATTATAAACATGTGGCAGAAAGT
AGGAAAAGCAATGTATGCCCCTCCCATCAGTGGACAAATTAGATGTTCATCAAATATTACAGG
GCTGCTATTAACAAGAGATGGTGGTAATAGCAACAATGAGTCCGAGATCTTCAGACCTGGAGG
AGGAGATATGAGGGACAATTGGAGAAGTGAATTATATAAATATAAAGTAGTAAAAATTGAACC
ATTAGGAGTAGCACCCACCAAGGCAAAGAGAAGAGTGGTGCAGAGAGAAAAAAGAGCAGTGGG
AATAGGAGCTTTGTTCCTTGGGTTCTTGGGAGCAGCAGGAAGCACTATGGGCGCAGCCTCAAT
GACGCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCTCCA
GGCAAGAATCCTGGCTGTGGAAAGATACCTAAAGGATCAACAGCTCCTGGGGATTTGGGGTTG
CTCTGGAAAACTCATTTGCACCACTGCTGTGCCTTGGAATGCTAGTTGGAGTAATAAATCTCT
GGAACAGATTTGGAATCACACGACCTGGATGGAGTGGGACAGAGAAATTAACAATTACACAAG
CTTAATACACTCCTTAATTGAAGAATCGCAAAACCAGCAAGAAAAGAATGAACAAGAATTATT
GGAATTAGATAAATGGGCAAGTTTGTGGAATTGGTTTAACATAACAAATTGGCTGTGGTATAT
AAAATTATTCATAATGATAGTAGGAGGCTTGGTAGGTTTAAGAATAGTTTTTGCTGTACTTTC
TATAGTGAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGAGACAGATC
CATTCGATTAGTGAACGGATCCTTGGCACTTATCTGGGACGATCTGCGGAGCCTGTGCCTCTT
CAGCTACCACCGCTTGAGAGACTTACTCTTGATTGTAACGAGGATTGTGGAACTTCTGGGACG
CAGGGGGTGGGAAGCCCTCAAATATTGGTGGAATCTCCTACAGTATTGGAGTCAGGAACTAAA
GAATAGTGCTGTTAGCTTGCTCAATGCCACAGCCATAGCAGTAGCTGAGGGGACAGATAGGGT
TATAGAAGTAGTACAAGGAGCTTGTAGAGCTATTCGCCACATACCTAGAAGAATAAGACAGGG
CTTGGAAAGGATTTTGCTATAAGATGGGTGGCAAGTGGTCAAAAAGTAGTGTGATTGGATGGC
CTACTGTAAGGGAAAGAATGAGACGAGCTGAGCCAGCAGCAGATAGGGTGGGAGCAGCATCTC
GAGACCTGGAAAAACATGGAGCAATCACAAGTAGCAATACAGCAGCTACCAATGCTGCTTGTG
CCTGGCTAGAAGCACAAGAGGAGGAGGAGGTGGGTTTTCCAGTCACACCTCAGGTACCTTTAA
GACCAATGACTTACAAGGCAGCTGTAGATCTTAGCCACTTTTTAAAAGAAAAGGGGGGACTGG
AAGGGCTAATTCACTCCCAAAGAAGACAAGATATCCTTGATCTGTGGAT
(CXCR4_V3_OPT_2B in NC1802_ENV region):
SEQ ID NO 37
GTGTGGTCCATAGTAATCATAGAATATAGGAAAATATTAAGACAAAGAAAAATAGACAGGTTA
ATTGATAGACTAAGAGAAAGAGCAGAAGACAGTGGCAATGAGAGTGAAGGAGAAATATCAGCA
CTTGTGGAGATGGGGGTGGAGATGGGGCACCATGCTCCTTGGGATGTTGATGATCTGTAGTGC
TACAGAAAAATTGTGGGTCACAGTCTATTATGGGGTACCTGTGTGGAAGGAAGCAACCACCAC
TCTATTTTGTGCATCAGATGCTAAAGCATATGATACAGAGGTACATAATGTTTGGGCCACACA
TGCCTGTGTACCCACAGACCCCAACCCACAAGAAGTAGTATTGGTAAATGTGACAGAAAATTT
TAACATGTGGAAAAATGACATGGTAGAACAGATGCATGAGGATATAATCAGTTTATGGGATCA
AAGCCTAAAGCCATGTGTAAAATTAACCCCACTCTGTGTTAGTTTAAAGTGCACTGATTTGAA
GAATGATACTAATACCAATAGTAGTAGCGGGAGAATGATAATGGAGAAAGGAGAGATAAAAAA
CTGCTCTTTCAATATCAGCACAAGCATAAGAGGTAAGGTGCAGAAAGAATATGCATTTTTTTA
TAAACTTGATATAATACCAATAGATAATGATACTACCAGCTATAAGTTGACAAGTTGTAACAC
CTCAGTCATTACACAGGCCTGTCCAAAGGTATCCTTTGAGCCAATTCCCATACATTATTGTGC
CCCGGCTGGTTTTGCGATTCTAAAATGTAATAATAAGACGTTCAATGGAACAGGACCATGTAC
AAATGTCAGCACAGTACAATGTACACATGGAATTAGGCCAGTAGTATCAACTCAACTGCTGTT
AAATGGCAGTCTAGCAGAAGAAGAGGTAGTAATTAGATCTGTCAATTTCACGGACAATGCTAA
AACCATAATAGTACAGCTGAACACATCTGTAGAAATTAATTGTACAAGACCCAACAACAATAC
AAGAAAAAGTATACGTATAGGACCAGGGAGAGCATTTTATGCAACAGGAAAAATAATAGGAGA
TATAAGACAAGCACATTGTAACATTAGTAGAGCAAAATGGAATAACACTTTAAAACAGATAGC
TAGCAAATTAAGAGAACAATTTGGAAATAATAAAACAATAATCTTTAAGCAATCCTCAGGAGG
GGACCCAGAAATTGTAACGCACAGTTTTAATTGTGGAGGGGAATTTTTCTACTGTAATTCAAC
ACAACTGTTTAATAGTACTTGGTTTAATAGTACTTGGAGTACTGAAGGGTCAAATAACACTGA
AGGAAGTGACACAATCACCCTCCCATGCAGAATAAAACAAATTATAAACATGTGGCAGAAAGT
AGGAAAAGCAATGTATGCCCCTCCCATCAGTGGACAAATTAGATGTTCATCAAATATTACAGG
GCTGCTATTAACAAGAGATGGTGGTAATAGCAACAATGAGTCCGAGATCTTCAGACCTGGAGG
AGGAGATATGAGGGACAATTGGAGAAGTGAATTATATAAATATAAAGTAGTAAAAATTGAACC
ATTAGGAGTAGCACCCACCAAGGCAAAGAGAAGAGTGGTGCAGAGAGAAAAAAGAGCAGTGGG
AATAGGAGCTTTGTTCCTTGGGTTCTTGGGAGCAGCAGGAAGCACTATGGGCGCAGCCTCAAT
GACGCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCTCCA
GGCAAGAATCCTGGCTGTGGAAAGATACCTAAAGGATCAACAGCTCCTGGGGATTTGGGGTTG
CTCTGGAAAACTCATTTGCACCACTGCTGTGCCTTGGAATGCTAGTTGGAGTAATAAATCTCT
GGAACAGATTTGGAATCACACGACCTGGATGGAGTGGGACAGAGAAATTAACAATTACACAAG
CTTAATACACTCCTTAATTGAAGAATCGCAAAACCAGCAAGAAAAGAATGAACAAGAATTATT
GGAATTAGATAAATGGGCAAGTTTGTGGAATTGGTTTAACATAACAAATTGGCTGTGGTATAT
AAAATTATTCATAATGATAGTAGGAGGCTTGGTAGGTTTAAGAATAGTTTTTGCTGTACTTTC
TATAGTGAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGAGACAGATC
CATTCGATTAGTGAACGGATCCTTGGCACTTATCTGGGACGATCTGCGGAGCCTGTGCCTCTT
CAGCTACCACCGCTTGAGAGACTTACTCTTGATTGTAACGAGGATTGTGGAACTTCTGGGACG
CAGGGGGTGGGAAGCCCTCAAATATTGGTGGAATCTCCTACAGTATTGGAGTCAGGAACTAAA
GAATAGTGCTGTTAGCTTGCTCAATGCCACAGCCATAGCAGTAGCTGAGGGGACAGATAGGGT
TATAGAAGTAGTACAAGGAGCTTGTAGAGCTATTCGCCACATACCTAGAAGAATAAGACAGGG
CTTGGAAAGGATTTTGCTATAAGATGGGTGGCAAGTGGTCAAAAAGTAGTGTGATTGGATGGC
CTACTGTAAGGGAAAGAATGAGACGAGCTGAGCCAGCAGCAGATAGGGTGGGAGCAGCATCTC
GAGACCTGGAAAAACATGGAGCAATCACAAGTAGCAATACAGCAGCTACCAATGCTGCTTGTG
CCTGGCTAGAAGCACAAGAGGAGGAGGAGGTGGGTTTTCCAGTCACACCTCAGGTACCTTTAA
GACCAATGACTTACAAGGCAGCTGTAGATCTTAGCCACTTTTTAAAAGAAAAGGGGGGACTGG
AAGGGCTAATTCACTCCCAAAGAAGACAAGATATCCTTGATCTGTGGAT
(US1 v3 in NC1802_ENV region):
SEQ ID NO 38
GTGTGGTCCATAGTAATCATAGAATATAGGAAAATATTAAGACAAAGAAAAATAGACAGGTTA
ATTGATAGACTAAGAGAAAGAGCAGAAGACAGTGGCAATGAGAGTGAAGGAGAAATATCAGCA
CTTGTGGAGATGGGGGTGGAGATGGGGCACCATGCTCCTTGGGATGTTGATGATCTGTAGTGC
TACAGAAAAATTGTGGGTCACAGTCTATTATGGGGTACCTGTGTGGAAGGAAGCAACCACCAC
TCTATTTTGTGCATCAGATGCTAAAGCATATGATACAGAGGTACATAATGTTTGGGCCACACA
TGCCTGTGTACCCACAGACCCCAACCCACAAGAAGTAGTATTGGTAAATGTGACAGAAAATTT
TAACATGTGGAAAAATGACATGGTAGAACAGATGCATGAGGATATAATCAGTTTATGGGATCA
AAGCCTAAAGCCATGTGTAAAATTAACCCCACTCTGTGTTAGTTTAAAGTGCACTGATTTGAA
GAATGATACTAATACCAATAGTAGTAGCGGGAGAATGATAATGGAGAAAGGAGAGATAAAAAA
CTGCTCTTTCAATATCAGCACAAGCATAAGAGGTAAGGTGCAGAAAGAATATGCATTTTTTTA
TAAACTTGATATAATACCAATAGATAATGATACTACCAGCTATAAGTTGACAAGTTGTAACAC
CTCAGTCATTACACAGGCCTGTCCAAAGGTATCCTTTGAGCCAATTCCCATACATTATTGTGC
CCCGGCTGGTTTTGCGATTCTAAAATGTAATAATAAGACGTTCAATGGAACAGGACCATGTAC
AAATGTCAGCACAGTACAATGTACACATGGAATTAGGCCAGTAGTATCAACTCAACTGCTGTT
AAATGGCAGTCTAGCAGAAGAAGAGGTAGTAATTAGATCTGTCAATTTCACGGACAATGCTAA
AACCATAATAGTACAGCTGAACACATCTGTAGAAATTAATTGTACAAGACCCAGCAACAATAC
AAGAAAAAGTATACATATAGGACCAGGGAGAGCATTTTATACAACAGGAAATATAATAGGAGA
TATAAGACAAGCACATTGTAACATTAGTAGAGCAAAATGGAATAACACTTTAAAACAGATAGC
TAGCAAATTAAGAGAACAATTTGGAAATAATAAAACAATAATCTTTAAGCAATCCTCAGGAGG
GGACCCAGAAATTGTAACGCACAGTTTTAATTGTGGAGGGGAATTTTTCTACTGTAATTCAAC
ACAACTGTTTAATAGTACTTGGTTTAATAGTACTTGGAGTACTGAAGGGTCAAATAACACTGA
AGGAAGTGACACAATCACCCTCCCATGCAGAATAAAACAAATTATAAACATGTGGCAGAAAGT
AGGAAAAGCAATGTATGCCCCTCCCATCAGTGGACAAATTAGATGTTCATCAAATATTACAGG
GCTGCTATTAACAAGAGATGGTGGTAATAGCAACAATGAGTCCGAGATCTTCAGACCTGGAGG
AGGAGATATGAGGGACAATTGGAGAAGTGAATTATATAAATATAAAGTAGTAAAAATTGAACC
ATTAGGAGTAGCACCCACCAAGGCAAAGAGAAGAGTGGTGCAGAGAGAAAAAAGAGCAGTGGG
AATAGGAGCTTTGTTCCTTGGGTTCTTGGGAGCAGCAGGAAGCACTATGGGCGCAGCCTCAAT
GACGCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCTCCA
GGCAAGAATCCTGGCTGTGGAAAGATACCTAAAGGATCAACAGCTCCTGGGGATTTGGGGTTG
CTCTGGAAAACTCATTTGCACCACTGCTGTGCCTTGGAATGCTAGTTGGAGTAATAAATCTCT
GGAACAGATTTGGAATCACACGACCTGGATGGAGTGGGACAGAGAAATTAACAATTACACAAG
CTTAATACACTCCTTAATTGAAGAATCGCAAAACCAGCAAGAAAAGAATGAACAAGAATTATT
GGAATTAGATAAATGGGCAAGTTTGTGGAATTGGTTTAACATAACAAATTGGCTGTGGTATAT
AAAATTATTCATAATGATAGTAGGAGGCTTGGTAGGTTTAAGAATAGTTTTTGCTGTACTTTC
TATAGTGAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGAGACAGATC
CATTCGATTAGTGAACGGATCCTTGGCACTTATCTGGGACGATCTGCGGAGCCTGTGCCTCTT
CAGCTACCACCGCTTGAGAGACTTACTCTTGATTGTAACGAGGATTGTGGAACTTCTGGGACG
CAGGGGGTGGGAAGCCCTCAAATATTGGTGGAATCTCCTACAGTATTGGAGTCAGGAACTAAA
GAATAGTGCTGTTAGCTTGCTCAATGCCACAGCCATAGCAGTAGCTGAGGGGACAGATAGGGT
TATAGAAGTAGTACAAGGAGCTTGTAGAGCTATTCGCCACATACCTAGAAGAATAAGACAGGG
CTTGGAAAGGATTTTGCTATAAGATGGGTGGCAAGTGGTCAAAAAGTAGTGTGATTGGATGGC
CTACTGTAAGGGAAAGAATGAGACGAGCTGAGCCAGCAGCAGATAGGGTGGGAGCAGCATCTC
GAGACCTGGAAAAACATGGAGCAATCACAAGTAGCAATACAGCAGCTACCAATGCTGCTTGTG
CCTGGCTAGAAGCACAAGAGGAGGAGGAGGTGGGTTTTCCAGTCACACCTCAGGTACCTTTAA
GACCAATGACTTACAAGGCAGCTGTAGATCTTAGCCACTTTTTAAAAGAAAAGGGGGGACTGG
AAGGGCTAATTCACTCCCAAAGAAGACAAGATATCCTTGATCTGTGGAT
(US2 v3 in NC1802_ENV region):
SEQ ID NO 39
GTGTGGTCCATAGTAATCATAGAATATAGGAAAATATTAAGACAAAGAAAAATAGACAGGTTA
ATTGATAGACTAAGAGAAAGAGCAGAAGACAGTGGCAATGAGAGTGAAGGAGAAATATCAGCA
CTTGTGGAGATGGGGGTGGAGATGGGGCACCATGCTCCTTGGGATGTTGATGATCTGTAGTGC
TACAGAAAAATTGTGGGTCACAGTCTATTATGGGGTACCTGTGTGGAAGGAAGCAACCACCAC
TCTATTTTGTGCATCAGATGCTAAAGCATATGATACAGAGGTACATAATGTTTGGGCCACACA
TGCCTGTGTACCCACAGACCCCAACCCACAAGAAGTAGTATTGGTAAATGTGACAGAAAATTT
TAACATGTGGAAAAATGACATGGTAGAACAGATGCATGAGGATATAATCAGTTTATGGGATCA
AAGCCTAAAGCCATGTGTAAAATTAACCCCACTCTGTGTTAGTTTAAAGTGCACTGATTTGAA
GAATGATACTAATACCAATAGTAGTAGCGGGAGAATGATAATGGAGAAAGGAGAGATAAAAAA
CTGCTCTTTCAATATCAGCACAAGCATAAGAGGTAAGGTGCAGAAAGAATATGCATTTTTTTA
TAAACTTGATATAATACCAATAGATAATGATACTACCAGCTATAAGTTGACAAGTTGTAACAC
CTCAGTCATTACACAGGCCTGTCCAAAGGTATCCTTTGAGCCAATTCCCATACATTATTGTGC
CCCGGCTGGTTTTGCGATTCTAAAATGTAATAATAAGACGTTCAATGGAACAGGACCATGTAC
AAATGTCAGCACAGTACAATGTACACATGGAATTAGGCCAGTAGTATCAACTCAACTGCTGTT
AAATGGCAGTCTAGCAGAAGAAGAGGTAGTAATTAGATCTGTCAATTTCACGGACAATGCTAA
AACCATAATAGTACAGCTGAACACATCTGTAGAAATTAATTGCATAAGACCCAACAACAATAC
AAGAAAAAGTATACATATAGGACCAGGGAGAGCAATTTATGCAACAGGAGGCATAATAGGAGA
TATAAGACGAGCATATTGTAACATTAGTAGAGCAAAATGGAATAACACTTTAAAACAGATAGC
TAGCAAATTAAGAGAACAATTTGGAAATAATAAAACAATAATCTTTAAGCAATCCTCAGGAGG
GGACCCAGAAATTGTAACGCACAGTTTTAATTGTGGAGGGGAATTTTTCTACTGTAATTCAAC
ACAACTGTTTAATAGTACTTGGTTTAATAGTACTTGGAGTACTGAAGGGTCAAATAACACTGA
AGGAAGTGACACAATCACCCTCCCATGCAGAATAAAACAAATTATAAACATGTGGCAGAAAGT
AGGAAAAGCAATGTATGCCCCTCCCATCAGTGGACAAATTAGATGTTCATCAAATATTACAGG
GCTGCTATTAACAAGAGATGGTGGTAATAGCAACAATGAGTCCGAGATCTTCAGACCTGGAGG
AGGAGATATGAGGGACAATTGGAGAAGTGAATTATATAAATATAAAGTAGTAAAAATTGAACC
ATTAGGAGTAGCACCCACCAAGGCAAAGAGAAGAGTGGTGCAGAGAGAAAAAAGAGCAGTGGG
AATAGGAGCTTTGTTCCTTGGGTTCTTGGGAGCAGCAGGAAGCACTATGGGCGCAGCCTCAAT
GACGCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCTCCA
GGCAAGAATCCTGGCTGTGGAAAGATACCTAAAGGATCAACAGCTCCTGGGGATTTGGGGTTG
CTCTGGAAAACTCATTTGCACCACTGCTGTGCCTTGGAATGCTAGTTGGAGTAATAAATCTCT
GGAACAGATTTGGAATCACACGACCTGGATGGAGTGGGACAGAGAAATTAACAATTACACAAG
CTTAATACACTCCTTAATTGAAGAATCGCAAAACCAGCAAGAAAAGAATGAACAAGAATTATT
GGAATTAGATAAATGGGCAAGTTTGTGGAATTGGTTTAACATAACAAATTGGCTGTGGTATAT
AAAATTATTCATAATGATAGTAGGAGGCTTGGTAGGTTTAAGAATAGTTTTTGCTGTACTTTC
TATAGTGAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAGAGACAGATC
CATTCGATTAGTGAACGGATCCTTGGCACTTATCTGGGACGATCTGCGGAGCCTGTGCCTCTT
CAGCTACCACCGCTTGAGAGACTTACTCTTGATTGTAACGAGGATTGTGGAACTTCTGGGACG
CAGGGGGTGGGAAGCCCTCAAATATTGGTGGAATCTCCTACAGTATTGGAGTCAGGAACTAAA
GAATAGTGCTGTTAGCTTGCTCAATGCCACAGCCATAGCAGTAGCTGAGGGGACAGATAGGGT
TATAGAAGTAGTACAAGGAGCTTGTAGAGCTATTCGCCACATACCTAGAAGAATAAGACAGGG
CTTGGAAAGGATTTTGCTATAAGATGGGTGGCAAGTGGTCAAAAAGTAGTGTGATTGGATGGC
CTACTGTAAGGGAAAGAATGAGACGAGCTGAGCCAGCAGCAGATAGGGTGGGAGCAGCATCTC
GAGACCTGGAAAAACATGGAGCAATCACAAGTAGCAATACAGCAGCTACCAATGCTGCTTGTG
CCTGGCTAGAAGCACAAGAGGAGGAGGAGGTGGGTTTTCCAGTCACACCTCAGGTACCTTTAA
GACCAATGACTTACAAGGCAGCTGTAGATCTTAGCCACTTTTTAAAAGAAAAGGGGGGACTGG
AAGGGCTAATTCACTCCCAAAGAAGACAAGATATCCTTGATCTGTGGAT
(1_R5_v3_B consensus):
SEQ ID NO 40
ATTACACAGGCCTGTCCAAAGGTATCCTTTGAGCCAATTCCCATACATTATTGTGCCCCGGCT
GGTTTTGCGATTCTAAAATGTAATAATAAGACGTTCAATGGAACAGGACCATGTACAAATGTC
AGCACAGTACAATGTACACATGGAATTAGGCCAGTAGTATCAACTCAACTGCTGTTAAATGGC
AGTCTAGCAGAAGAAGAGGTAGTAATTAGATCTGTCAATTTCACGGACAATGCTAAAACCATA
ATAGTACAGCTGAACACATCTGTAGAAATTAATTGTACAAGACCCAACAACAATACAAGAAAA
AGTATACATATAGGACCAGGGAGAGCATTTTATGCAACAGGAGAAATAATAGGAGATATAAGA
CAAGCACATTGTAACATTAGTAGAGCAAAATGGAATAACACTTTAAAACAGATAGCTAGCAAA
TTAAGAGAACAATTTGGAAATAATAAAACAATAATCTTTAAGCAATCCTCAGGAGGGGACCCA
GAAATTGTAACGCACAGTTTTAATTGTGGAGGGGAATTTTTCTACTGTAATTCAACACAACTG
TTTAATAGTACTTGGTTTAATAGTACTTGGAGTACTGAAGGGTCAAATAACACTGAAGGAAGT
GACACAATCACCCTCCCATGCAGAATAAAACAAATTATAAACATGTGGCAGAAAGTAGGAAAA
GCAATGTATGCCCCTCCCATCAGTGGACAAATTAGATGTTCATCAAATATTACAGGGCTGCTA
TTAACAAGAGATGGTGGTAATAGCAACAATGAGTCCGAGATCTTCAGACCTGGAGGAGGAGAT
ATGAGGGACAATTGGAGAAGTGAATTATATAAATATAAAGTAGTAAAAATTGAACCATTAGGA
GTAGCACCCACCAAGGCAAAGAGAAGAGTGGTGCAGAGAG
(2_R4_V3_OPT_2B):
SEQ ID NO 41
ATTACACAGGCCTGTCCAAAGGTATCCTTTGAGCCAATTCCCATACATTATTGTGCCCCGGCT
GGTTTTGCGATTCTAAAATGTAATAATAAGACGTTCAATGGAACAGGACCATGTACAAATGTC
AGCACAGTACAATGTACACATGGAATTAGGCCAGTAGTATCAACTCAACTGCTGTTAAATGGC
AGTCTAGCAGAAGAAGAGGTAGTAATTAGATCTGTCAATTTCACGGACAATGCTAAAACCATA
ATAGTACAGCTGAACACATCTGTAGAAATTAATTGTACAAGACCCAACAACAATACAAGAAAA
AGTATACGTATAGGACCAGGGAGAGCATTTTATGCAACAGGAAAAATAATAGGAGATATAAGA
CAAGCACATTGTAACATTAGTAGAGCAAAATGGAATAACACTTTAAAACAGATAGCTAGCAAA
TTAAGAGAACAATTTGGAAATAATAAAACAATAATCTTTAAGCAATCCTCAGGAGGGGACCCA
GAAATTGTAACGCACAGTTTTAATTGTGGAGGGGAATTTTTCTACTGTAATTCAACACAACTG
TTTAATAGTACTTGGTTTAATAGTACTTGGAGTACTGAAGGGTCAAATAACACTGAAGGAAGT
GACACAATCACCCTCCCATGCAGAATAAAACAAATTATAAACATGTGGCAGAAAGTAGGAAAA
GCAATGTATGCCCCTCCCATCAGTGGACAAATTAGATGTTCATCAAATATTACAGGGCTGCTA
TTAACAAGAGATGGTGGTAATAGCAACAATGAGTCCGAGATCTTCAGACCTGGAGGAGGAGAT
ATGAGGGACAATTGGAGAAGTGAATTATATAAATATAAAGTAGTAAAAATTGAACCATTAGGA
GTAGCACCCACCAAGGCAAAGAGAAGAGTGGTGCAGAGAG

### II—Amplification Methods

In some embodiments, the present disclosure provides methods that use the aforementioned oligonucleotides as amplification primers to amplify regions of the HIV genome, in particular regions that encode the V3 region of the env gene. As discussed in more detail below, in some embodiments the primers are used in nested PCR methods for the amplification and detection or sequencing of the HIV env gene or fragments thereof. In some embodiments, the aforementioned oligonucleotides are used as amplification primers to amplify regions of the HIV genome present in one or more nucleic acids in a sample taken from a patient (e.g., a plasma sample). In some embodiments, the aforementioned oligonucleotides are used as amplification primers to amplify regions of the HIV genome present in one or more of the aforementioned nucleic acid controls.

### Preparation of RNA

In some embodiments, the disclosed methods may involve some level of RNA preparation. Indeed, the template for an amplification reaction (e.g., a PCR reaction) is typically DNA and the target HIV material to be analyzed is typically expressed viral RNA (or a control RNA transcript as described herein). As a result, the starting template material for the amplification reaction will often be cDNA which was generated from purified RNA including RNA from viruses. The RNA preparation step may be performed far removed from the actual amplification step, for example, in another laboratory, or at a much earlier time; however, in some embodiments the RNA isolation and preparation of the cDNA may occur in conjunction with the amplification step of the methods.

When an RNA preparation step is included in the disclosed methods, the method of RNA preparation can be any method of RNA preparation that produces enzymatically manipulatable mRNA. For example, the RNA can be isolated by using the guanidinium isothiocyanate-ultracentrifugation method, the guanidinium and phenol-chloroform method, the lithium chloride-SDS-urea method or poly A+/mRNA from tissue lysates using oligo(dT) cellulose method, e.g., see Schildkraut et al., J. Mol. Biol. 4, 430-433 (1962); Chomczynski and Sacchi, Anal. Biochem. 162:156 (1987); Auffray and Rougeon, Eur. J. Biochem. 107:303-314 (1980); Aviv and Leder, Proc. Natl. Acad. Sci. USA 69, 1408-1412 (1972); and Sambrook et al., Selection of poly A+RNA in “Molecular Cloning”, Vol. 1, 7.26-7.29 (1989).

RNA can be isolated from any desired cell or cell type and from any organism, including mammals, such as mouse, rat, rabbit, dog, cat, monkey, and human, as well as other non-mammalian animals, such as fish or amphibians, as well as plants and even prokaryotes, such as bacteria. Thus, the DNA used in the method can also be from any organism, such as that disclosed for RNA.

Generation of cDNA

In some embodiments, disclosed methods involve cDNA preparation. The cDNA preparation step may be performed far removed from the actual amplification step, for example, in another laboratory, or at a much earlier time; however, in some embodiments the preparation of the cDNA may occur in conjunction with the amplification step of the methods.

When a cDNA preparation step is included in the disclosed methods, the method of cDNA preparation can be any method of cDNA preparation that produces enzymatically manipulatable cDNA. For example, the cDNA can be prepared by using, for example, random primers, poly-d(T) oligos, or NVd(T) oligos. For the purpose of data normalization, an equal amount of total RNA is typically used for cDNA synthesis. Many examples exist of performing reverse transcription to produce cDNA for use in PCR, including the following: Glisin et al., Biochemistry 13:2633-7 (1974); Ullrich et al., Science 196:1313 (1977); Chirgwin et al., Biochemistry 18:5294-9 (1979); Faulkner-Jones et al., Endocrinol. 133:2962-2972 (1993); and Gonda et al., Mol. Cell Biol. 2:617-624 (1982).

Reverse transcriptases from any source (native or recombinant) may be used in the practice of the present disclosure. Suitable reverse transcriptases include, but are not limited to, those from Moloney murine leukemia virus (M-MLV), human T-cell leukemia virus type I (HTLV-I), bovine leukemia virus (BLV), Avian Sarcoma Leukemia Viruses (ASLV) including Rous Sarcoma Virus (RSV) and Avian Myeloblastosis Virus (AMV), human immunodeficiency virus (HIV), cauliflower mosaic virus, Saccharomyces, Neurospora, Drosophila, primates, and rodents. See, for example, U.S. Pat. Nos. 4,663,290 and 6,063,60; Grandgenett, et al., Proc. Nat. Acad. Sci. (USA) 70:230-234 (1973), Gerard, DNA 5:271-279 (1986), Kotewicz, et al., Gene 35:249-258 (1985), Tanese et al., Proc. Natl. Acad. Sci. (USA) 82:4944-4948 (1985), Roth et al., J. Biol. Chem. 260:9326-9335 (1985), Michel et al., Nature 316:641-643 (1985), Akins et al., Cell 47:505-516 (1986) and EMBO J. 4:1267-75 (1985), and Fawcett, Cell 47:1007-1015 (1986); Shinnick et al., Nature 293:543-548 (1981); Seiki et al., Proc. Natl. Acad. Sci. USA 80:3618-3622 (1983); Rice et al., Virology 142:357-77 (1985); Schwartz et al., Cell 32:853-869 (1983); Larder et al., EMBO J. 6:3133-3137 (1987); Farmerie et al., Science 236:305-308 (1987); Barr et al., Biotechnology 5:486-489 (1987)); Tanese et al., J. Virol. 59:743-745 (1986); Hansen et al., J. Biol. Chem. 262:12393-12396 (1987); Sonigo et al., Cell 45:375-85 (1986); Takatsuji et al., Nature 319:240-243 (1986); Toh et al., Nature 305:827-829 (1983)); Alexander et al., J. Virol. 61:534-542 (1987); and Yuki et al., Nucl. Acids Res. 14:3017-3030 (1986).

### Amplification Reaction

The use of oligonucleotide sequences of the present disclosure as primers to amplify HIV env target sequences in test samples is not limited to any particular nucleic acid amplification technique or any particular modification thereof. In fact, the inventive oligonucleotide sequences can be employed in any of a variety of nucleic acid amplification methods well-known in the art (see, for example, Kimmel and Berger, Methods Enzymol. 152: 307-316 (1987); Sambrook et al., “Molecular Cloning: A Laboratory Manual”, 1989, 2^{nd} Ed., Cold Spring Harbour Laboratory Press: New York, N.Y.; “Short Protocols in Molecular Biology”, Ausubel (Ed.), 2002, 5^{th} Ed., John Wiley & Sons: Secaucus, N.J.).

Such nucleic acid amplification methods include, but are not limited to, the Polymerase Chain Reaction (or PCR, described, for example, in “PCR Protocols: A Guide to Methods and Applications”, Innis (Ed.), 1990, Academic Press: New York; “PCR Strategies”, Innis (Ed.), 1995, Academic Press: New York; “Polymerase chain reaction: basic principles and automation in PCR: A Practical Approach”, McPherson et al. (Eds.), 1991, IRL Press: Oxford; Saiki et al., Nature 324:163 (1986); and U.S. Pat. Nos. 4,683,195, 4,683,202 and 4,889,818, each of which is incorporated herein by reference in its entirety); and reverse transcriptase polymerase chain reaction (or RT-PCR, described in, for example, U.S. Pat. Nos. 5,322,770 and 5,310,652).

The PCR (or polymerase chain reaction) technique is well-known in the art and has been disclosed, for example, in Mullis and Faloona, Methods Enzymol., 155:350-355 (1987). In its simplest form, PCR is an in vitro method for the enzymatic synthesis of specific DNA sequences, using two primers that hybridize to opposite strands and flank the region of interest in the target DNA. A plurality of reaction cycles, each cycle comprising: a denaturation step, an annealing step, and a polymerization step, results in the exponential accumulation of a specific DNA fragment, see for example, “PCR Protocols: A Guide to Methods and Applications”, Innis (Ed.), 1990, Academic Press: New York; “PCR Strategies”, Innis (Ed.), 1995, Academic Press: New York; “Polymerase chain reaction: basic principles and automation in PCR: A Practical Approach”, McPherson et al. (Eds.), 1991, IRL Press: Oxford; Saiki et al., Nature 324:163-166 (1986). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq) which are available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR (RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 30 to about 50.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually about 90° C. to about 94° C. for a period of time ranging from about 3 to about 120 seconds, usually from about 5 to about 30 seconds. In some embodiments, the first cycle is preceded by an elongated denaturation step ranging from about 1 to 10 minutes, usually from about 2 to 5 minutes.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 40° C. to about 75° C., usually from about 45° C. to about 70° C., and more usually from about 48° C. to about 62° C. Annealing conditions are generally maintained for a period of time ranging from about 15 seconds to about 30 minutes, usually from about 30 seconds to about 1 minute.

Following annealing of primer to template DNA or during annealing of primer to template DNA, the reaction mixture is subjected to conditions sufficient to provide for polymerization of nucleotides to the primer's end in a such manner that the primer is extended in a 5′ to 3′ direction using the DNA to which it is hybridized as a template (i.e., conditions sufficient for enzymatic production of primer extension product). To achieve primer extension conditions, the temperature of the reaction mixture is typically raised to a temperature ranging from about 65° C. to about 75° C., usually from about 68° C. to about 72° C., and maintained at that temperature for a period of time ranging from about 15 seconds to about 20 minutes, usually from about 30 seconds to about 5 minutes, and more usually for 2 minutes. In some embodiments, the final extension step is followed by an elongated extension step ranging from about 1 to 20 minutes, usually from about 2 to 10 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610. Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

In some embodiments, the PCR reaction is a “kinetic PCR” (kPCR) or “kinetic RT-PCR” (kRT-PCR) reaction, which are also referred to as “real-time PCR” and “real-time RT-PCR,” respectively. These methods involve detecting PCR products via a probe that provides a signal (typically a fluorescent signal) that is related to the amount of amplified product in the sample. Examples of commonly used probes used in kPCR and kRT-PCR include the following probes: TAQMAN® probes, Molecular Beacons probes, SCORPION® probes, and SYBR® Green probes. Briefly, TAQMAN® probes, Molecular Beacons, and SCORPION® probes each have a fluorescent reporter dye (also called a “fluor”) attached on or around the 5′ end of the probes and a quencher moiety attached on or around the 3′ end of the probes. In the unhybridized state, the proximity of the fluor and the quench molecules prevents the detection of fluorescent signal from the probe. During PCR, when the polymerase replicates a template on which a probe is bound, the 5′-nuclease activity of the polymerase cleaves the probe at a site between the fluor and quencher thus, increasing fluorescence with each replication cycle. SYBR® Green probes bind double-stranded DNA and upon excitation emit light; thus as PCR product accumulates, fluorescence increases.

In some embodiments, the PCR reaction is used in a “single-plex” PCR assay. “Single-plex” refers to a single assay that is not carried out simultaneously with any other assays. Single-plex assays include individual assays that are carried out sequentially.

In some embodiments, the PCR reaction is used in a “multiplex” PCR assay. The term “multiplex” refers to multiple assays that are carried out simultaneously, in which detection and analysis steps are generally performed in parallel. Within the context of the present disclosure, a multiplex assay will include the use of the primers, alone or in combination with additional primers to identify, for example, an HIV virus variant along with one or more additional HIV variants or other viruses.

In some embodiments, a first amplification step amplifies a region of a target gene. In some embodiments the amplification product is less than about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 250, 225, 200, 175 or 150 nucleotides long.

### Nested PCR

In some embodiments, oligonucleotides of SEQ ID NOS: 1-33 can be used in a “nested” PCR reaction to accurately amplify the V3 region of the HIV env gene. A “nested” PCR reaction refers to a two-step specific amplification of a target nucleic acid. In the first amplification step, a segment of nucleic acid is amplified using a first (outer) primer set. In the second amplification step, a second (inner) primer set is used to further amplify a segment of the segment that was amplified in the first step. Both first- and second-step primer sets will flank the target nucleic acid. As a result, the final amplified product is obtained within the frame of the segment that was amplified in the first step. The present disclosure may be used in conjunction with any nested PCR system known to those of skill in the art to generate an amplified target nucleic acid sequence. In some embodiments, the methods can employ a reverse transcription step to produce cDNA, a first amplification step performed with a first (outer) primer set which is specific to a target sequence, a second amplification step performed with a second (inner) primer set on all or a portion of the first amplification mixture, and optionally a detection or sequencing step to determine the presence or sequence of the target sequence.

In some embodiments, forward primers for a first amplification step of nested PCR of the V3 region of the HIV env gene are set forth in SEQ ID NOS: 1, 2, 3, 12, 16, 17 or 30 or any active fragments or combinations thereof. In some embodiments, reverse primers for a first amplification step of nested PCR of the V3 region of the HIV env gene are set forth in SEQ ID NOS: 4, 5, 6, 13, 18-21, or 31 or any active fragment thereof. In some embodiments, the forward and reverse primers comprise an outer primer set for the first amplification step of the V3 region of the HIV env gene. In some embodiments, this outer primer set comprises Primer sets 1, 3, 5, 7, or 9.

A second round of PCR amplification, e.g., in order to ensure PCR specificity for the target sequence of interest, can be performed on the amplification product of the first amplification step. For instance, the amplicon, e.g., the V3 region of the HIV env gene, can be amplified in a PCR reaction with an inner primer set.

In some embodiments, forward primers for a second amplification step of nested PCR of the V3 region of the HIV env gene are set forth in SEQ ID NOS: 7, 14, or 22-25 or any active fragments or combinations thereof. In some embodiments, reverse primers for a second amplification step of nested PCR of the of the V3 region of the HIV env gene are set forth in SEQ ID NOS: 8-11, 15, 26-29, or 32-33 or any active fragments or combinations thereof.

In some embodiments, the forward and reverse primers comprise an inner primer set for the second amplification step of the V3 region of the HIV env gene. In some embodiments, this inner primer set comprises Primer sets 2, 4, 6, 8 or 10.

In some embodiments, the inner primer set is used to amplify an amplification product of the outer primer set. In some embodiments, the primers of the inner primer set are homologous to the amplification product of the outer primer set. In some embodiments, the inner primer set are homologous to the amplification product of the outer primer set and overlap with the outer primer set. In some embodiments, the inner primer set are homologous to the amplification product of the outer primer set and do not overlap with the outer primer set.

In some embodiments, the outer primer set comprises Primer sets 1, 5, or 9 and the inner primer set comprises Primer sets 2, 6, or 10. In some embodiments, the outer primer set comprises Primer sets 3 or 7 and the inner primer set comprises Primer sets 4 or 8.

### Detection of Amplification Products

Amplification products generated using the oligonucleotides and methods of the present disclosure may be detected using a variety of methods known in the art.

In some embodiments, amplification products may simply be detected using agarose gel electrophoresis and visualization by ethidium bromide staining and exposure to ultraviolet (UV) light.

In some embodiments, the presence of a specific genotype can be shown by restriction enzyme analysis. For example, a specific nucleotide polymorphism can result in a nucleotide sequence comprising a restriction site which is absent from the nucleotide sequence of another tropic variant. Additionally or alternately, a specific nucleotide polymorphism can result in the elimination of a nucleotide sequence comprising a restriction site which is present in the nucleotide sequence of another tropic variant.

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally and then hybridized to target DNA under conditions which permit hybridization only if a perfect match is found, e.g., see Saiki et al., Nature 324:163 (1986); Saiki et al., Proc. Natl Acad. Sci USA 86:6230 (1989); and Wallace et al., Nucl. Acids Res. 6:3543 (1979). Such specific oligonucleotide hybridization techniques may be used for the simultaneous detection of several nucleotide changes in different polymorphic regions of DNA. For example, oligonucleotides having nucleotide sequences of specific tropic variants are attached to a hybridizing membrane and this membrane is then hybridized with labeled sample nucleic acid. Analysis of the hybridization signal will then reveal the identity of the nucleotides of the sample nucleic acid. Alternatively unlabeled sample nucleic acid may be immobilized and contacted with labeled oligonucleotides that hybridize selectively with specific tropic variants.

Real-time pyrophosphate DNA sequencing is yet another approach to detection of HIV sequence variations conferring tropism, e.g., see Alderborn et al., Genome Research, 10(8):1249-1258 (2000). Additional methods include, for example, PCR amplification in combination with denaturing high performance liquid chromatography (dHPLC), e.g., see Underhill et al., Genome Research, 7(10):996-1005 (1997).

In some embodiments, any of a variety of sequencing reactions known in the art can be used to directly sequence at least a portion of amplified DNA and detect tropic variants. The sequence can be compared with the sequences of known tropic variants to determine which one(s) are present in the sample. Exemplary sequencing reactions include those based on techniques developed by Maxam and Gilbert, Proc. Natl. Acad. Sci USA, 74:560 (1977) or Sanger, Proc. Nat. Acad. Sci 74:5463 (1977). It is also contemplated that any of a variety of automated sequencing procedures may be utilized when performing the subject assays, e.g., see Venter et al., Science, 291:1304-1351 (2001); Lander et al., Nature, 409:860-921 (2001), including sequencing by mass spectrometry, e.g., see U.S. Pat. No. 5,547,835 and PCT Patent Publication No. WO 94/16101 and WO 94/21822; U.S. Pat. No. 5,605,798 and PCT Patent Application No. PCT/US96/03651; Cohen et al., Adv. Chromatogr. 36:127-162 (1996); and Griffin et al., Appl. Biochem. Biotechnol. 38:147-159 (1993). It will be evident to one skilled in the art that, for some embodiments, the occurrence of only one, two or three of the nucleic acid bases need be determined in the sequencing reaction. Yet other sequencing methods are disclosed, e.g., in U.S. Pat. Nos. 5,580,732; 5,571,676; 4,863,849; 5,302,509; PCT Patent Application Nos. WO 91/06678 and WO 93/21340; Canard et al., Gene 148:1-6 (1994); Metzker et al., Nucleic Acids Research 22:4259-4267 (1994) and U.S. Pat. Nos. 5,740,341 and 6,306,597.

In some embodiments, PCR-based amplification products may be sequenced using, but not limited to, Next Generation Sequencing techniques. These include, but are not limited to, Single Molecule Real Time (SMRT) sequencing, DNA Nanoball Sequencing, Massively Parallel Signature Sequencing (MPSS), Heliscope Single Molecule Sequencing, Illumina Dye Sequencing, Polony Sequencing, Ion Semiconductor Sequencing, SOLiD Sequencing and 454 Pyrosequencing. In other embodiments, sequencing techniques still under development may be used to sequence the amplification products of the present disclosure. Such sequencing techniques include, but are not limited to, Transmission Electron Microscopy DNA Sequencing, Nanopore Sequencing, RNAP Sequencing, Microfluidic Sanger Sequencing, Sequencing by Hybridization, In vitro Virus High-Throughout Sequencing, Mass Spectrometry Sequencing, and Tunneling Currents DNA Sequencing. Next generation sequencing methods are disclosed, e.g., in Liu et al., J. Biomed. Biotechnol. 251364 (2012); Cheng et al., Front Genet. 4:150 (2013); Pavlopoulos et al., BioData Min. 6(1):13 (2013); Chen et al., Biomed Res Int. 2013:901578 (2103), PCT Application Nos. WO 2012/148497; WO 2011/096926; WO 2007/092538; and U.S. Patent Application Nos. US 2012/0330566; US 2010/0120098; and US 2013/0122494.

### Exemplary Assay

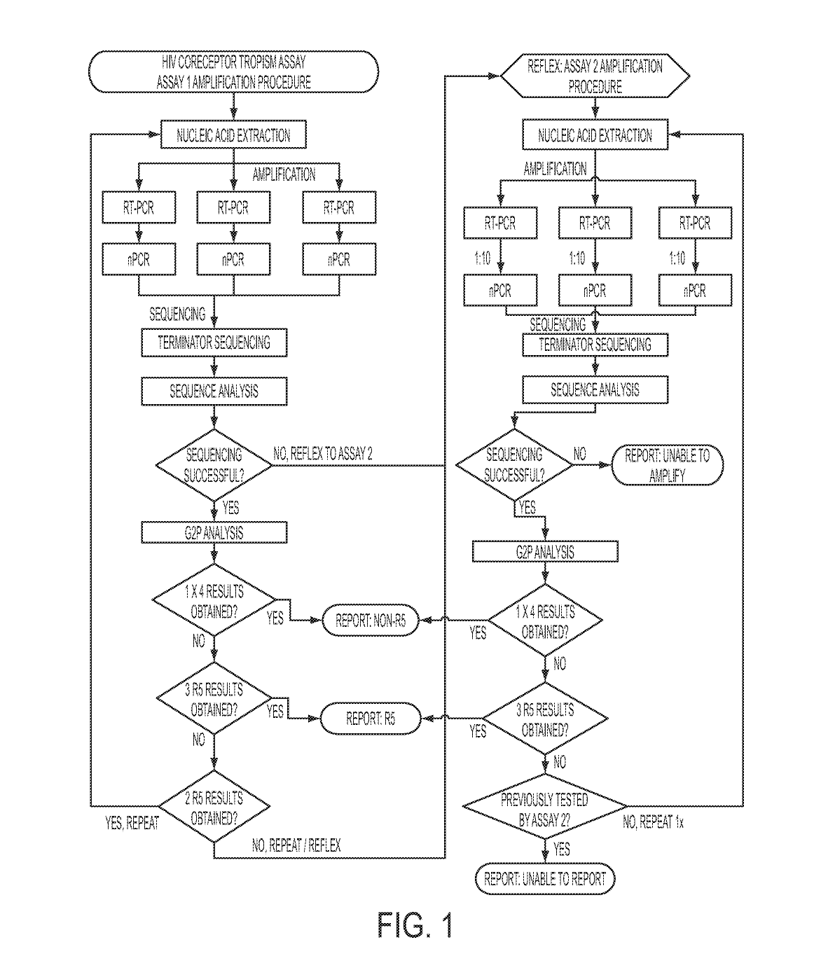

In some embodiments, the present disclosure provides an assay to accurately amplify the V3 region of the HIV env gene comprising methods for the amplification and sequencing of the V3 region of the HIV env gene described herein. The Exemplary Assay presented herein presents a method of in vitro nucleic acid amplification and sequencing for the determination of human immunodeficiency virus type 1 (HIV-1) coreceptor tropism (CCR5 or CXCR4). This method, outlined in FIG. 1, is intended to identify patients appropriate for coreceptor antagonist therapies such as SELZENTRY, a CCR5 antagonist, by analysis of EDTA plasma from HIV-1 infected individuals. The results of this method are useful to predict response to coreceptor entry inhibitors as an aid in the clinical management of HIV-1 infected patients. In some embodiments, the present disclosure provides a method to identify patients appropriate for other coreceptor antagonist therapies, e.g., without limitation Vicriviroc (Schering-Plough), Aplaviroc (GlaxoSmithKline), Cenicriviroc (Takeda) and PRO 140 (Cytodyn).

RNA is extracted from HIV-1 positive human plasma and a first tropism assay is performed. Once sequence data is obtained, tropism prediction algorithms described herein are used to identify coreceptor usage. Samples that generate insufficient data to report coreceptor usage by the first tropism assay are processed further using the second tropism assay and sequence data is subject to tropism prediction algorithms to identify coreceptor usage.

In some embodiments, a first tropism assay comprises a first RNA extraction step, a first RT-PCR step, a first nested PCR step, and a first sequencing step. In some embodiments, a second tropism assay comprises a second RNA extraction step, a second RT-PCR step, a second nested PCR step, and a second sequencing step. RNA extraction, RT-PCR, nested PCR, and sequencing steps are performed according to methods described herein. In some embodiments, RT-PCR and nested PCR steps are performed in triplicate.

In some embodiments, a first RT-PCR step comprises contacting the RNA from the first RNA extraction step with a first set of outer primers consisting of Primer Set 1 or Primer Set 5.

In some embodiments, a first nested PCR step comprises contacting the product of the first RT-PCR step with a first set of inner primers consisting of Primer Set 2 or Primer Set 6. In some embodiments, the first set of inner primers are tagged. In some embodiments, the first set of inner primers are tagged with M13 tag sequences.

In some embodiments, a second RT-PCR step comprises contacting the RNA from the second RNA extraction step with a second set of outer primers consisting of Primer Set 3 or Primer Set 7.

In some embodiments, a second nested PCR step comprises contacting the product of the second RT-PCR step with a second set of inner primers consisting of Primer Set 4 or Primer Set 8. In some embodiments, the product of the second RT-PCR step is diluted. In some embodiments, the product of the second RT-PCR step is diluted 1:2, 1:5, 1:10, 1:20, or 1:100. In some embodiments, the first set of inner primers are tagged. In some embodiments, the first set of inner primers are tagged with M13 tag sequences.

### III—Kits

In some embodiments, the present disclosure provides kits comprising materials useful for the amplification and detection or sequencing of the V3 region of the HIV env gene according to methods described herein. The inventive kits may be used by diagnostic laboratories, experimental laboratories, or practitioners.

Materials and reagents useful for the detection or sequencing of the V3 region of the HIV env gene according to the present disclosure may be assembled together in a kit. In some embodiments, an inventive kit comprises at least one inventive primer set, and optionally, reverse transcription and/or amplification reaction reagents. In some embodiments, a kit comprises reagents which render the procedure specific. Thus, a kit intended to be used for the detection of a particular HIV coreceptor tropism (e.g., CCR5 or CXCR4) preferably comprises primer sets described herein that can be used to amplify a particular HIV target sequence of interest. A kit intended to be used for the multiplex detection of a plurality of HIV target sequences and/or other viruses preferably comprises a plurality of primer sets (optionally in separate containers) described herein that can be used to amplify HIV target sequences described herein.

Suitable reverse transcription/amplification reaction reagents that can be included in an inventive kit include, for example, one or more of: buffers; enzymes having reverse transcriptase and/or polymerase activity; enzyme cofactors such as magnesium or manganese; salts; nicotinamide adenide dinuclease (NAD); and deoxynucleoside triphosphates (dNTPs) such as, for example, deoxyadenosine triphosphate; deoxyguanosine triphosphate, deoxycytidine triphosphate and deoxythymidine triphosphate, biotinylated dNTPs, suitable for carrying out the amplification reactions.

Depending on the procedure, the kit may further comprise one or more of: wash buffers and/or reagents, hybridization buffers and/or reagents, labeling buffers and/or reagents, and detection means. The buffers and/or reagents included in a kit are preferably optimized for the particular amplification/detection technique for which the kit is intended. Protocols for using these buffers and reagents for performing different steps of the procedure may also be included in the kit.

Furthermore, the kits may be provided with an internal control as a check on the amplification procedure and to prevent occurrence of false negative test results due to failures in the amplification procedure. An optimal control sequence is selected in such a way that it will not compete with the target nucleic acid sequence in the amplification reaction (as described above).

Kits may also contain reagents for the isolation of nucleic acids from biological specimen prior to amplification and/or for the purification or separation of HIV before nucleic acid extraction.

As mentioned above, kits that also contain one or more nucleic acid controls (e.g., a mixture of the aforementioned nucleic acid controls). In some embodiments, the one or more nucleic acid controls are DNA based (e.g., DNA vectors). In some embodiments, the one or more nucleic acid controls are RNA based (e.g., RNA transcript).

The reagents may be supplied in a solid (e.g., lyophilized) or liquid form. The kits of the present disclosure optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial sale.

The kit may also comprise instructions for using the amplification reaction reagents and primer sets or primer/probe sets according to the present disclosure. Instructions for using the kit according to one or more methods of the present disclosure may comprise instructions for processing the biological sample, extracting nucleic acid molecules, and/or performing the test; instructions for interpreting the results as well as a notice in the form prescribed by a governmental agency (e.g., FDA) regulating the manufacture, use or sale of pharmaceuticals or biological products.

### IV—Diagnostic and Therapeutic Methods

In some embodiments, the present disclosure provides diagnostic methods which involve performing the amplification and sequencing steps to determine the CCR5 and CXCR4 coreceptor status for a patient (e.g., to determine whether a patient is infected with CXCR4 coreceptor variants). It will be appreciated that the diagnosing, amplification and sequencing steps could be performed by separate entities and that the present disclosure provides methods which involve a step of sequencing an amplicon that was previously generated in accordance with the inventive methods and also methods that involve a step of diagnosing based on a sequence that was previously generated in accordance with the inventive methods.

In some embodiments, the present disclosure provides a diagnostic test used for any HIV entry inhibitor that requires coreceptor tropism determination before administering a treatment. The detection of coreceptor tropism indicates a course of HIV treatment. Drugs used to treat HIV infection based upon tropism include, but are not limited to, Maraviroc/SELZENTRY (Pfizer), Vicriviroc (Schering-Plough), Aplaviroc (GlaxoSmithKline), Cenicriviroc (Takeda) and PRO 140 (Cytodyn). In some embodiments, the detection of CCR5 and CXCR4 coreceptor variants can be used to determine a course of HIV treatment. In some embodiments, detection of CCR5 indicates a positive prognosis for using the drug Maraviroc/SELZENTRY (Pfizer) to treat HIV infection. In other embodiments, detection of CXCR4 indicates that treatment with the drug Maraviroc/SELZENTRY will be less effective. The same methods may also be used with other anti-HIV therapeutics that are more effective with CCR5 coreceptor variants (or conversely with anti-HIV therapeutics that are more effective with CXCR4 coreceptor variants) (e.g., Vicriviroc (Schering-Plough), Aplaviroc (GlaxoSmithKline), Cenicriviroc (Takeda) and PRO 140 (Cytodyn)).

### EXAMPLES

### Example 1

### HIV Coreceptor Tropism Assay

The following example presents a method of in vitro nucleic acid amplification and sequencing for the determination of human immunodeficiency virus type 1 (HIV-1) coreceptor tropism (CCR5 or CXCR4). This method, outlined in FIG. 1, is intended to identify patients appropriate for coreceptor antagonist therapies such as SELZENTRY, a CCR5 antagonist, by analysis of EDTA plasma from HIV-1 infected individuals. The results of this method should be used in conjunction with clinical and other laboratory information to predict response to coreceptor entry inhibitors as an aid in the clinical management of HIV-1 infected patients.

Nucleic acids are extracted from HIV-1 positive human EDTA plasma with the QIAamp Viral RNA Mini Kit (Qiagen). Sequencing is performed in triplicate using one of two primer-specific procedures, Assay 1 or Assay 2.

Assay 1

Assay 1 begins with the extracted HIV-1 RNA template that is reverse transcribed and then amplified by polymerase chain reaction (RT-PCR).

RT-PCR amplification utilizes General Purpose Reagents (Siemens) and gp160 outer primers to amplify the complete env gene (2,844 base pairs, bp). Subsequently, nested PCR amplification is performed (414 bp) with M13-tailed gp120-specific inner primers to incorporate universal M13 sequencing sites on the ends of the amplicon.

RNA Extraction Procedure

The centrifuge and rotor are pre-chilled to 2° to 8° C. Plasma samples and controls are thawed at room temperature for approximately 15 minutes. 500 μL of plasma or controls is pipetted into a labeled 1.5 mL sterile screw cap Sarstedt tube. Tubes are centrifuged at 23,500×g for 1 hour at 2° to 8° C. 350 μL of the supernatant is aspirated and discarded. The remaining volume is gently mixed by tapping the bottom of the tube. Viral RNA is extracted using the Qiagen Viral RNA Extraction kit. Extracts are stored at −60° to −80° C., or RT-PCR is performed immediately.

RT-PCR Procedure

100 μM stocks of V3 Loop RT-PCR primer solutions (EMF1, EMF1-2, EMR1, and EMR1-2) are thawed for 10 minutes at room temperature. Stocks are then vortexed briefly and spun to collect at the bottom of the tube. 30 μM working RT-PCR primers are prepared in nuclease-free water.

Master Mix 1 is prepared by mixing 12.40 μL Nuclease free water, 1.50 μL dNTP Solution, 1.00 μL DTT Solution, 0.15 μL EMF1-1 (SEQ ID NO: 2) (30 μM), 0.15 μL EMF1-2 (SEQ ID NO: 3) (30 μM), 0.15 μL EMR1-1 (SEQ ID NO: 5) (30 μM), 0.15 μL EMR1-2 (SEQ ID NO: 6) (30 μM), and 0.50 μL RNase Inhibitor in a final volume of 16 μL per reaction.

Master Mix 2 is prepared by mixing 10.00 μL RT-PCR Buffer, 0.50 μL Rnase Inhibitor, 1.00 μL SuperScript III RT Enzyme, and 2.50 μL DNA Polymerase in a total volume of 14 μL per reaction.

Master Mix 1 is briefly vortexed and then 16 μL Master Mix 1 is mixed with 10 μL sample in a PCR plate. The plate is placed in the thermocycler. For the reverse transcription reaction, the thermocycler is run at 90° C. for 2 minutes, 52° C. for 20 minutes, and 94° C. for 2 minutes. After 5 minutes at 52° C., the thermocycler program is paused and 14 μL Master Mix 2 is added to each sample. The program is then resumed. After the reverse transcription reaction completes, the thermocycler proceeds directly into the amplification reaction. The amplification reaction is 37 cycles comprising of a 30 second denaturation step at 94° C. followed by a 30 second annealing step at 62° C. followed by a 2 minute extension step at 68° C. After the final cycle, there is a final extension step of 2 minutes at 68° C. followed by an infinite hold at 4° C.

Nested PCR

Subsequently, nested PCR amplification is performed (414 bp) with M13-tailed gp120-specific primers to incorporate universal M13 sequencing sites on the ends of the amplicon. Each double-stranded DNA amplicon generated with the Assay 1 procedure is sequenced using the Applied Biosystems BigDye Terminator v3.1 Cycle Sequencing Kit in conjunction with M13 universal sequencing primers to generate the nucleotide sequence of the V3 region of envelope protein gp120.

100 μM stocks of V3 Loop nPCR primer solutions (6957F with 5′ M13F_BP tag, 7371-2R with 5′ M13R_BP tag, 7371-3R with 5′ M13R_BP tag, and 7371-4R-M1 with 5′ M13R_BP tag) are thawed for 10 minutes at room temperature. Stocks are then vortexed briefly and spun to collect at the bottom of the tube. 1 μM working nPCR primers are prepared in nuclease-free water.

nPCR Master Mix is prepared by mixing 24.00 μL Nuclease free water, 5.00 μL 10×PCR Buffer II, 1.00 μL 10 mM dNTP Mix, 2.00 μL 25 mM MgCl2 Solution, 5.00 μL Forward Primer 6957F (SEQ ID NO: 7) with 5′ M13F_BP tag (SEQ ID NO: 34) (1 μM), 2.50 μL Reverse Primer 7371-2R (SEQ ID NO: 9) with 5′ M13R_BP tag (SEQ ID NO: 35) (1 μM), 2.50 μL Reverse Primer 7371-3R (SEQ ID NO: 10) with 5′ M13R_BP tag (1 μM), 2.50 μL Reverse Primer 7371-4R-M1 (SEQ ID NO: 11) with 5′ M13R_BP tag (1 μM), 0.50 μL AmpliTaq Gold (5 U/μL) to a final volume of 45 μL per sample. For each sample, 45 μL nPCR Master Mix and 5 μL RT PCR product is added to a well of a PCR plate.

The nested PCR reaction begins with a 5 minute denaturation step at 94° C. followed by 35 cycles comprised of a 30 second denaturation step at 94° C. followed by a 30 second annealing step at 57° C. followed by a 2 minute extension step at 72° C. After the final cycle, there is a final extension step of 2 minutes at 72° C. followed by an infinite hold at 4° C.

After the nested PCR reaction has completed, 8 μL of ExoSAP-IT is added to each reaction and mixed by pipeting. The plate is returned to the thermocycler and incubated at 37° C. for 15 minutes followed by 80° C. for 15 minutes followed by an infinite hold at 4° C.

Assay 2

Samples that generate insufficient data to report coreceptor usage by the Assay 1 procedure are processed further using the Assay 2 procedure.

The HIV-1 RNA template is reverse transcribed and amplified using a One-Step RT-PCR Kit (Qiagen) to amplify a 717 bp amplicon. A nested PCR is performed with the RT PCR product using primers that amplify 337 base pairs and incorporate M13 sequencing sites into the amplicon. The double-stranded DNA amplicon is sequenced using the Applied Biosystems BigDye Terminator v3.1 Cycle Sequencing Kit in conjunction with M13 universal sequencing primers.

RNA Extraction Procedure

A fresh aliquot of each sample is extracted with the QIAamp Viral RNA Mini Kit. Plasma samples or controls are thawed at room temperature for approximately 15 minutes. RNA is extracted from 140 μL of sample or control using Qiagen Viral RNA Extraction kit. Extracts are stored at −60° to −80° C., or proceed immediately to RT-PCR.

RT-PCR Procedure

100 μM stocks of V3 Loop RT-PCR primer solutions (Italian_RT_{—}1F, Italian_RT_{—}1R) are thawed for 10 minutes at room temperature. Tubes are vortexed briefly and spin to collect at the bottom of the tube. 10 μM working RT PCR primers are prepared in nuclease free water.

1-Step MasterMix is prepared by mixing 2.50 μL RNAse free water, 5.00 μL 5×PCR Buffer, 1.00 μL 10 mM dNTP Mix, 2.50 μL Forward Primer Italian_RT_{—}1F (SEQ ID NO: 16) (10 μM), 2.50 μL Reverse Primer Italian_RT_{—}1R (SEQ ID NO: 18) (10 μM), 1.00 μL Enzyme Mix and 0.50 μL RNase inhibitor 20 U/μL to a final volume of 15 μL per sample. 1-Step MasterMix is gently vortexed. 15 μL1-Step MasterMix is mixed with 10 μL sample in a PCR plate.

The plate is placed in the thermocycler. For the reverse transcription reaction, the thermocycler is run at 50° C. for 30 minutes, and 95° C. for 15 minutes. After the reverse transcription reaction completes, the thermocycler proceeds directly into the amplification reaction. The amplification reaction is 45 cycles comprised of a 30 second denaturation step at 94° C. followed by a 30 second annealing step at 52° C. followed by a 2 minute extension step at 72° C. After the final cycle, there is a final extension step of 10 minutes at 72° C. followed by an infinite hold at 4° C.

Nested PCR

100 μM stocks of V3 Loop nPCR primer solutions (Italian_Seq_{—}1F and Italian_Seq_{—}1R) are thawed for 10 minutes at room temperature. Tubes are vortexed briefly and spin to collect at the bottom of the tube. 1 μM working RT PCR primers are prepared in nuclease free water.

nPCR Master Mix is prepared by mixing 26.50 μL Nuclease free water, 5.00 μL 10×PCR Gold Buffer, 1.00 μL GeneAmp® dNTP Blend, 10 mM (Applied Biosystems), 2.00 μL 25 mM MgCl2 Solution, 5.00 μL Forward Primer Italian_Seq_{—}1F (SEQ ID NO: 22) with 5′ M13F_BP tag (SEQ ID NO: 34) (1 μM), 5.00 μL Reverse Primer Italian_Seq_{—}1R (SEQ ID NO: 26) with 5′ M13R_BP tag (SEQ ID NO: 35) (1 μM), 0.50 μL AmpliTaq Gold (5 U/μL) to a final volume of 45 μL per sample. For each sample, 45 μL nPCR Master Mix and 5 μL of a 1:10 dilution of the RT PCR product is added to a well of a PCR plate.

The nested PCR reaction begins with a 5 minute denaturation step at 94° C. followed by 35 cycles comprised of a 30 second denaturation step at 94° C. followed by a 30 second annealing step at 48° C. followed by a 2 minute extension step at 72° C. After the final cycle, there is a final extension step of 2 minutes at 72° C. followed by an infinite hold at 4° C.

After the nested PCR reaction has completed, 8 μL of ExoSAP-IT is added to each reaction and mixed by pipeting. The plate is returned to the thermocycler and incubated at 37° C. for 15 minutes followed by 80° C. for 15 minutes followed by an infinite hold at 4° C.

Sequencing Reaction

The sequencing reaction master mix is prepared by mixing 11.50 μL Molecular Grade Type 1 Water, 3.50 μL 5× Sequencing Buffer, and 1.00 μL BDT Sequencing Mix v3.1 for a total of 16 μL per reaction.

In the wells of a PCR reaction plate, 2 μL 2 μM Forward Primer M13F BP (SEQ ID NO: 34), 2 μL 2 μM Reverse Primer M13R BP (SEQ ID NO: 35), 2 μL of the nested PCR reaction product, and 16 μL sequencing reaction master mix are mixed.

The sequencing PCR reaction begins with a 1 minute denaturation step at 96° C. followed by 40 cycles comprising a 30 second denaturation step at 96° C. followed by a 10 second annealing step at 50° C. followed by a 3 minute extension step at 60° C. After the final cycle, there is an infinite hold at 4° C.

Following amplification, the amplification product is purified. A mixture of 500 μL of 3M NaOAc and 500 μL of 125 μM EDTA is prepared and 4 μL of the mixture is added to each reaction. 50 μL 100% ethanol is then added to each reaction. The PCR reaction plate is then incubated at room temperature for 15 minutes and centrifuged at 3,000×g for 30 minutes at 2 to 8° C. Each reaction is then washed with 150 μL of 70% ethanol and spun at 2,000×g for 10 minutes at 2 to 8° C. After the supernatant is removed, the PCR plate is then inverted and spun at 700×g for 1 minute at 2 to 8° C. to remove excess ethanol.

The fluorescently labeled chain termination fragments from the sequencing reaction are analyzed using the 3500×L Genetic Analyzer (Life Technologies). Each forward and reverse sequence quality value (QV) score is reviewed. A QV score of at least 25 for the sequence is required to continue analysis against the reference sequence.

Double-stranded sequence data are aligned to a reference sequence that is a consensus sequence of HIV-1 subtype B compiled from sequences in the HIV database from Los Alamos National Lab. Each edited consensus sequence is exported for interpretation using the geno2pheno[coreceptor] algorithm with optimized parameters. A False Positive Rate (FPR) cutoff of less than or equal to 10% for non-R5 prediction is utilized. Three CCR5 (R5) co-receptor tropism results are required to report R5 tropism to confirm a candidate for CCR5 antagonist therapy. A single non-R5 (CXCR4 co-receptor) result excludes a patient as a candidate for CCR5 antagonist therapy and is reported as non-R5 HIV-1.

Results

Sample Preparation Evaluation

Based on sample volume availability, randomly selected clinical samples from three clinical trials were used for this study. All samples had viral loads of at least 1000 copies/mL. Forty-five (45) unique samples were tested using Assay 1. The reportable tropism report rate was calculated for each condition.

Two extraction volumes were used for each sample. A 140 μL Extraction Volume of plasma was extracted using the Qiagen QIAamp Viral RNA Mini Kit (Qiagen) following manufacturer's standard protocol. A500 μL Extraction Volume of plasma was centrifuged for one hour. Then, 350 μL of supernatant was discarded and the remaining volume (˜150 μL) was used to resuspend the pellet, which was extracted with the Qiagen QIAamp Viral RNA Mini Kit.

The acceptance criteria was ≧88% reportable tropism result rate obtained when the viral load is ≧1000 copies/mL (Svicher et. al. “Performance of genotypic tropism testing in clinical practice using the enhanced sensitivity version of Trofile as reference assay: results from the OSCAR Study Group.” New Microbiologica. 33, 195-206, 2010; Sanchez et al. “Performance of Genotypic Algorithms for Predicting HIV-1 Tropism Measured against the Enhanced-Sensitivity Trofile Coreceptor Tropism Assay.” Journal of Clinical Microbiology, 4135-4139, November 2010). The assay using Assay 1 was 100% (45/45) successful at 500 μL and 96% (43/45) successful at 140 μL extraction volumes.

Analytical Accuracy

Analytical accuracy was evaluated using a panel created from a stock solution of cultured supernatant from the HIV-1 infected 8E5/LAV cell line. The panel consisted of five samples diluted in negative K3EDTA plasma to HIV-1 target-concentrations of 625, 1250, 2000, 2500, and 1,000,000 copies/mL. Nucleic acids for all samples were extracted according using 140 μL extraction volume. The samples were amplified and sequenced using Assay 1. Each panel member was analyzed in 21 replicates. 21 sequences were obtained at 2500 and 1,000,000 copies/mL. 19 sequences were obtained at 2000 copies/mL. 17 sequences were obtained at 1250 copies/mL. 7 sequences were obtained at 625 copies/mL.

DNA sequencing accuracy was determined with respect to a reference sequence for the gp120 v3 loop region. The alignment highlights positions different from the reference sequence in related sequences; including the BRU isolate (LAV-1) sequence K02013, HXB2 genomic reference sequence K03455, and a consensus sequence created from B subtype gp120 V3 loop region sequences from the Los Alamos National Laboratory (LANL) HIV Sequence Database. The percent error rate per 100 bps (base pairs) sequenced was calculated with respect to the reference sequence (HIV-1, 8E5).

All replicates generating reportable sequence at each level were aligned with the reference sequence. The acceptance criteria was accuracy of ≧98% (Product Insert, TRUGENE HIV-1 Genotyping Kit (IVD), PN: 1047425, Rev B, 2010-08). Each position in the alignment either was determined to be 100% consistent with the reference sequence across all replicates or was determined to include at least one mismatch. No strictly mismatched basecalls were detected. One sequence position for the 10e6 panel member included an ambiguous basecall that was denoted as a partial mismatch (hetero match). The linear sum of positions sequenced across all replicates with sequence was 9,180 base positions. The percentage of basecalls that perfectly matched the reference sequence was 99.99%.

Analytical Sensitivity, Subtype B

Analytical sensitivity panel members were prepared from a randomly selected HIV-1 positive sample characterized as Group M subtype B of unknown tropism determination. The stock HIV-1 sample was diluted in basepool plasma to defined target concentrations (1000, 700, 500, 300, 250, 200, and 100 copies/mL). The acceptance criteria was analytical sensitivity at or less than 1000 RNA copies/mL, as defined by a reportable tropism result rate of ≧95%.

Panel members analyzed by Assay 1 used the 500 μL extraction volume process, and panel members analyzed by Assay 2 used the 140 μL extraction volume process.

To determine analytical sensitivity using Assay 1, a four-member panel was created with target concentrations of 1000, 700, 500, and 300 copies/mL. The study consisted of five (5) plates that generated 70 results. Based on 100% detection with this panel, a second set of panel members was prepared with additional target concentrations of 300, 250, 200, and 100 copies/mL. The study consisted of five (5) plates that generated 70 results. For Assay 1, reportable tropism results were obtained at 100% detection at 100 copies/mL.

To determine analytical sensitivity using Assay 2, one panel member was created from a stock solution of cultured supernatant from the HIV-1 infected 8E5/LAV cell line to a target concentration of 700 copies/mL. The study consisted of two (2) plates that generated 20 results. For Assay 2, reportable tropism results were obtained at 100% detection at 700 copies/mL.

Analytical Sensitivity, Non-B Subtypes

Analytical Sensitivity for the HIV Coreceptor Tropism Assay was evaluated for non-B subtypes. Each subtype lab panel (LP4) member was diluted in basepool to a target concentration of 1.0E+04 copies/mL. Non-B subtypes evaluated were: A, C, D, F, G, H, CRF_{—}01 (AE), and CRF_{—}02 (AG), consisting of either tropism value (R5, non-R5).

Panel members analyzed by Assay 1 used the 500 μL extraction volume process, and panel members analyzed by Assay 2 used the 140 μL extraction volume process.

Subtype sensitivity for the assay using Assay 1 across subtypes A, C, G, AE, and AG at 1000 copies/mL was 100% (50/50). Sensitivity for subtype H at 1500 copies/mL was 90% (9/10). Subtype F was not detected by Assay 1.

Subtype sensitivity for the assay using Assay 2 across subtypes A, C, D, F, G, and AG at 1000 copies/mL was 100% (60/60). Sensitivity for subtype H at 1000 copies/mL was 80% (8/10); sensitivity for subtype AE at 1000 copies/mL 10% (1/10).

Analytical Specificity, Negatives

A test panel consisting of twenty-two (22) unique HIV negative plasma samples from volunteer donors was tested. Panel members analyzed by the Assay 1 used the 500 μL extraction volume process, and panel members analyzed by the Assay 2 used the 140 μL extraction volume process. The acceptance criteria was no detection of HIV in HIV negative samples. No HIV V3 loop sequence was present in the HIV-negative samples tested.

Analytical Specificity, Interfering Substances

Analytical specificity for the amplification procedure in the presence of interfering substances was assessed. The study consisted of two groups of interfering substances:
(1) Endogenous Substances: Triglycerides, Albumin, Hemoglobin, Direct Bilirubin, Indirect Bilirubin
(2) Non-HIV Disease States: HCV, HBV, Multiple Myeloma (MM), Systemic Lupus Erythematosus (SLE) positive, Anti-nuclear Antibody (ANA) positive, Waldenstrom's Cyroglobulinema positive

Panel members were created by spiking each interfering substance individually into a stock source of HIV-1 of unknown tropism determination, resulting in a final HIV-1 concentration of 700 copies/mL. Panel members were then extracted using the 500 μL extraction volume process and tested using Assay 1. Assays were performed in triplicate. The acceptance criteria was 100% reportable tropism result rate at 1000 copies/mL.

Samples containing endogenous substances (and in the presence of other diseases) yielded a reportable tropism result rate of 100% at 700 copies/mL. Endogenous substances and other diseases did not interfere with the performance of the assay.

Analytical Reproducibility

Analytical reproducibility studies were performed with Assay 1 and Assay 2. Study material was made from an HIV-1 subtype B sample of unknown tropism determination diluted to 700 copies/mL in basepool plasma. Sixty (60) 1-mL aliquots were prepared for each study. Assay 1 used the 500 μL extraction volume process, and Assay 2 used the 140 μL extraction volume process. Each study used ten (10) aliquots per run, two (2) runs per day, over three (3) days, for a total of sixty (60) results per study.

Between Run results show 95% confidence limits for detecting a reportable tropism result for Assay 1 and Assay 2 procedures, respectively. Between Day results show 95% confidence limits for detecting a reportable tropism result for Assay 1 and Assay 2 procedures, respectively. No statistically significant differences in tropism results and reportable tropism result rate were observed within day, between runs, and between days, based on the following:
(1) Confidence limits for reportable tropism results overlap for the two runs within a given day and for between runs across three days.
(2) Confidence limits for reportable tropism results overlap for between day.

Transcript Mixture Sensitivity

Mixture sensitivity studies were performed using Assay 1 and Assay 2. For both Assay 1 and Assay 2 studies, mixture sensitivity was determined by testing panel members with varying ratios of CXCR4:CCR5.

The test panel consisted of mixtures of transcribed RNA, comprised of 0%, 15%, 20%, and 100% CXCR4 in a sample, with a RNA concentration equal to 10⁶ copies/mL of HIV. For each study (Assay 1 and Assay 2) 42 replicates were generated, with 1 replicate of 0% and 100% CXCR4 and 20 replicates of 15% and 20% CXCR4. The acceptance criteria is detection of greater than or equal to 95% CXCR4 for samples containing 20% CXCR4.

Using Assay 1, CXCR4 is detected in the sample with 100% CXCR4. CXCR4 is not detected in the sample with 0% CXCR4. CXCR4 is detected in 100% of samples with 15% CXCR4. CXCR4 is detected in 95% of samples with 20% CXCR4.

Using Assay 2, CXCR4 is detected in the sample with 100% CXCR4. CXCR4 is not detected in the sample with 0% CXCR4. CXCR4 is detected in 45% of samples with 15% CXCR4. CXCR4 is detected in 100% of samples with 20% CXCR4.

### Example 2

### Clinical Validity Studies

The following example describes clinical validity studies performed on the HIV Coreceptor Tropism Assay described in Example 1. A total of 363 blinded clinical samples were received for testing.

Before the entire sample set was tested by the HIV Coreceptor Tropism Assay, an initial set of unblinded samples (N=82) was tested to determine the optimal FPR (False Positive Rate) to use for tropism calls. Once the optimal parameters for tropism calls were set, the remaining blinded samples were tested. After tropism results were obtained for all samples using HIV Coreceptor Tropism Assay, the remaining samples were unblinded. The overall sequencing success rate and concordance to Trofile ES tropism results were calculated.

A geno2pheno FPR cut-off for determination of tropism of 10% was chosen for this assay for two reasons. First, the recently published European Guidelines recommends an FPR cut-off of 10%. Second, a more conservative cut-off of 10% minimizes the risk that the assay will overcall CCR5. This approach is intended to minimize potential risk to patients, in that fewer patients with non-CCR5 virus would be classified as having CCR5 virus. Thus, fewer patients with non-CCR5 virus will be misclassified as eligible for treatment with CCR5 antagonist.

The full dataset was composed of 363 samples. Twelve samples were Quantity Not Sufficient (QNS) for repeat testing and were excluded from the final dataset for analysis (N=351). Twenty-five samples were Unable to Report (UTR). Overall, in the final analysis dataset (N=351) the success rate of the HIV Coreceptor Tropism Assay was 92.9% (i.e., 326/351 points had reportable results).

The analysis dataset for concordance to Trofile-ES was N=317 and was composed of all patients for whom tropism assay results were available from both Trofile-ES and the HIV Coreceptor Tropism Assay.

The results presented herein suggest the HIV Coreceptor Tropism Assay is a good alternative for determining HIV viral tropism. Concordance to Trofile ES was 79.2%. This is similar to levels of agreement seen in other studies comparing phenotypic with genotypic tropism assays, i.e., in the range of 80% (de Mendoza et al. (2008) “Performance of a population-based HIV-1 tropism phenotypic assay and correlation with V3 genotypic prediction tools in recent HIV-1 seroconverters” JAIDS 48(3):241-4; Poveda et al. (2009) “Design and validation of new genotypic tools for easy and reliable estimation of HIV tropism before using CCR5 antagonists” J. Antimicrob Chemoth. 63(5):1006-10; Sanchez et al. (2010) “Performance of genotypic algorithms for predicting HIV-1 tropism measured against the enhanced-sensitivity Trofile co-receptor tropism assay” J. Clin. Microbiol. 48(11):4135-9). The assay also had a 92.9% sequencing success rate, which is comparable to what has been seen with phenotyping assays at 80-95% (Gonzalez-Serna et al. “TROCAI (Tropism Co-receptor Assay Information): a New Phenotypic Tropism Test and Its Correlation with Trofile Enhanced Sensitivity and Genotypic Approaches” J. Clin. Microbiol. 48(12):4453-4458; Coakley et al. (2009) “Comparison of human immunodeficiency virus type 1 tropism profiles in clinical samples by the Trofile and MT-2 Assays” Antimicrob. Agents Ch. 53(11):4686-4693).

### Example 3

### Coreceptor Tropism Analysis of Patients

The following example describes screening of antiretroviral naïve subjects for treatment with MVC+ZDV/3TC in Russia using the HIV-1 Coreceptor Tropism Assay described in Example 1. The assay was utilized prospectively in this on going Phase 3b/4 study. Genotypic coreceptor tropism, protease and reverse transcriptase drug resistance mutations, and HIV-1 subtype were determined during screening for a multicenter, open label study of maraviroc, zidovudine and lamivudine twice daily for the treatment of antiretroviral naïve HIV-infected patients with R5 HIV-1 in Russia. Enrolled subjects were undergoing treatment with a combination of Combivir (zidovudine and lamivudine) and maraviroc as their first line HIV therapy. The efficacy and safety of this combination in a Russian population of patients over 18 years of age with viral loads greater than 1,000 RNA copies/mL who have never been treated with anti-HIV medicines was assessed as well as the performance of the sequencing-based assay.

Materials & Methods

Screening of antiretroviral naïve subjects for treatment with MVC+ZDV/3TC was completed using the HIV-1 Coreceptor Tropism Assay. The assay input was viral RNA extracted from plasma. Triplicate reverse transcription and PCR amplification reactions are performed, followed by dye-terminator DNA sequencing of the V3 loop of HIV-1 glycoprotein 120 to identify CCR5 coreceptor-utilizing viruses (R5). Tropism assay analytical parameters were previously determined using panels of in vitro-transcribed env gene clones, subtype B HIV-1 (8E5), and non-B subtype HIV-1 isolates. Analytical performance specifications included viral load sensitivity for B and non-B subtypes (300 and 1,000 RNA copies/mL), sequencing accuracy (99.99%), and clonal sequence mixture sensitivity (20%).

The V3 loop of gp120 was analyzed using the Geno2Pheno[coreceptor] algorithm (Lengauer et al., “Bioinformatics prediction of HIV coreceptor usage” Nat Biotechnol. 2007) with a false positive rate threshold of 10% as described in the European guidelines on the clinical management of HIV-1 tropism testing (The Lancet Infectious Diseases, Volume 11, Issue 5, Pages 394-407, May 2011). High sensitivity for CXCR4 coreceptor-utilizing dual or mixed HIV-1 was achieved through the requirement of confirmation by three R5 results to identify a patient appropriate for use of maraviroc. A single non-R5 result was sufficient to report non-R5 virus.

Protease (codons 1 to 99) and reverse transcriptase (codons 40 to 247) regions were assessed for drug resistance mutations and subtype determinations were made based on sequencing using the TRUGENE HIV-1 Genotyping Kit and OPENGENE DNA Sequencing System. Pair-wise best match analysis with clade specific reference sequences was utilized for subtype determinations. The reference sequence set was constructed using complete genome sequences in the Los Alamos HIV Sequence Database (www.hiv.lanl.gov) and majority consensus sequences created for those subtypes and circulating recombinant forms (CRF) with more than 5 available sequences.

Results

153 subjects in Russia were screened for the study using the HIV-1 Coreceptor Tropism Assay. 145 of 153 patient samples produced reportable coreceptor tropism data. The most common reason for failure to report was the presence of a mixture of quasispecies with insertions or deletions.

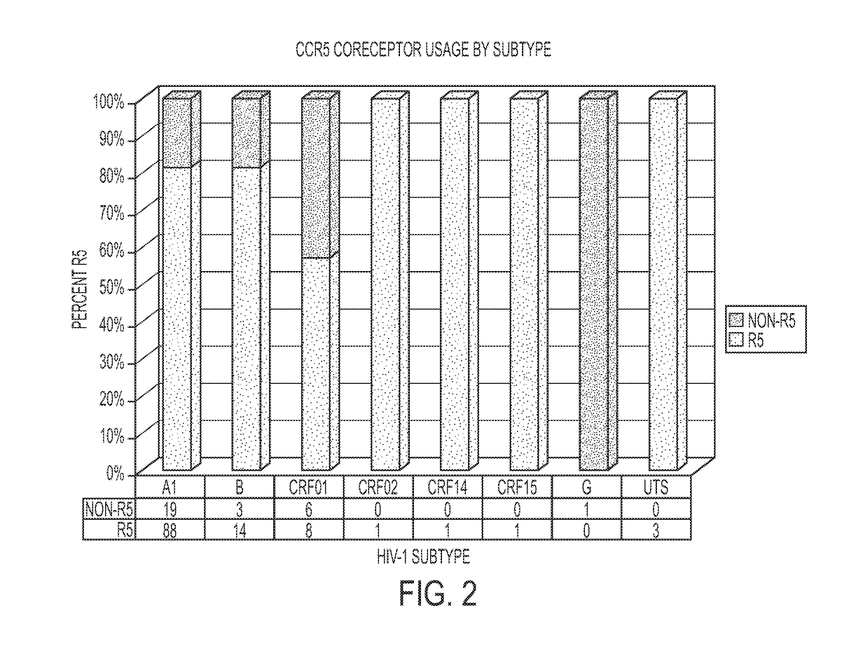

A1 was the most prevalent subtype (74.5%)—based on pol sequence analysis. Subtype B virus was reported for 11.1% of patients screened. Taken together CRF_{—}01 (AE), CRF_{—}15 (01B) and ambiguous CRF_{—}01 results including 01/A1, 01/15 and 01/15/A1 were determined for 10.5% of patients. Other reported clades included G, CRF_{—}02 (AG) and CRF_{—}14 (BG). Drug resistance mutations were reported in three cases (two occurrences of RT: K103N and one occurrence of PR: M461). R5 coreceptor usage was reported for 80% and non-R5 for 20% of patients after triplicate R5 confirmation. The prevalence of R5 usage was the same for A1 and B subtype viruses (82.2% and 82.4% respectively). Although the sample size was small, the viruses with CRF_{—}01 sequences in pol had a lower rate of R5 tropism usage reported (60.0%, 9/15). Results are shown in FIG. 2.

Positive Predictive Value (PPV) of the assay was defined in terms of the proportion of patients in the study responding to therapy and is shown below in Table 2. Patients with early termination unrelated to virological response were excluded from the analysis. Patients with early termination due to lack of virological response or relapse at or before week 12 were considered non-responders. Positive virological outcome was defined as greater than 2 log₁₀ decline in HIV-1 RNA copies/mL (VL) from baseline to week 12 or undetectable VL. Undetectable VL conditions defined as <50 and <400 were used to determine sensitivity of PPV to variation of the VL cutoff. Two-sided confidence intervals were calculated at 95% to determine upper and lower confidence levels for PPV (95% UCL and LCL).

TABLE 2

| Definition of | | | | | |
|---|---|---|---|---|---|
| Virological | # | # | | 95% | 95% |
| Response | Patients | Responders | PPV | LCL | UCL |
| Change in Viral | 91 | 88 | 96.7% | 90.7% | 99.3% |
| Load >2log10 or | | | | | |
| Viral Load <50 | | | | | |
| Change in Viral | 91 | 89 | 97.8% | 92.3% | 99.7% |
| Load >2log10 or | | | | | |
| Viral Load <400 | | | | | |

### CONCLUSIONS

The HIV-1 Coreceptor Tropism Assay achieved a genotyping success rate of 94.8% for this population of patients with a large proportion of non-B subtype HIV-1 infection. Coreceptor tropism results were generated within a time-frame similar to the commercially available TRUGENE HIV-1 Drug Resistance Assay. The mean times to result of 6.8 and 6.0 days respectively for tropism and drug resistance testing were nearly identical.

Subtype A1 viruses were found in 74.5% of subjects screened for this study. CCR5 coreceptor usage frequency was determined to be the same in A1 and B subtypes. The 20% rate of non-R5 coreceptor usage in these antiretroviral treatment-naïve individuals was found to be similar although slightly higher than the 17% reported using phenotypic tropism testing for the MERIT study at screening before randomization, and lower than the 29% after reclassification of a subset of screening samples post-randomization with enhanced sensitivity phenotyping (Cooper et al., J. Infect. Dis. 2010; 201:803-813).

The number of non-R5 results for the CRF_{—}01 viruses was double that of A1 and B subtypes. This was consistent with results published by Chalmet et al. (J. Infect. Dis. 2012; 205(2):174-184) in which CXCR4 use was found to be significantly higher in CRF_{—}01 infections than in infections with subtype B, A, C, or CRF02.

The primary endpoint for the study, proportion of subjects with plasma HIV-1 RNA <50 copies/mL, is achieved after 48 weeks, and a formal interim analysis is completed after 24 weeks of treatment. However, assessment of assay performance before week 24 is considered informative and minimizes the confounding effect from patient discontinuations due to reasons unrelated to virological response.

Early virological outcome data at week 12 were used for clinical performance assessment of the assay with respect to selecting patients who may benefit from therapy. Clinical performance of the assay for selecting patients who responded to therapy based on early virological response rate was determined to be >90.7% (PPV LCL 95%).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. (canceled)

2. A collection of primers for amplifying a V3 region of the HIV env genomic sequence or a portion thereof comprising primer sets selected from the group consisting of Primer Set 1, Primer Set 2, Primer Set 3, Primer Set 4, Primer Set 5, Primer Set 6, Primer Set 7, and Primer Set 8 wherein:
Primer Set 1 comprises a forward primer comprising SEQ ID NO: 1, or any active fragment thereof, and a reverse primer comprising SEQ ID NO: 4, or any active fragment thereof,
Primer Set 2 comprises a forward primer comprising SEQ ID NO: 7, or any active fragment thereof, and a reverse primer comprising SEQ ID NO: 8 or SEQ ID NO: 11, or any active fragments or combinations thereof,
Primer Set 3 comprises a forward primer comprising SEQ ID NO: 12, or any active fragment thereof, and a reverse primer comprising SEQ ID NO: 13, or any active fragment thereof, and
Primer Set 4 comprises a forward primer comprising SEQ ID NO: 14, or any active fragment thereof, and a reverse primer comprising SEQ ID NO: 15, or any active fragment thereof,
Primer Set 5 comprises a forward primer comprising SEQ ID NO: 2 or SEQ ID NO: 3, or any active fragments or combinations thereof, and a reverse primer comprising SEQ ID NO: 5 or SEQ ID NO: 6, or any active fragments or combinations thereof,
Primer Set 6 comprises a forward primer comprising SEQ ID NO: 7, or any active fragment thereof, and a reverse primer comprising SEQ ID NO: 9, SEQ ID NO: 10, or SEQ ID NO: 11, or any active fragment or combinations thereof,
Primer Set 7 comprises a forward primer comprising SEQ ID NO: 16 or SEQ ID NO: 17, or any active fragments or combinations thereof, and a reverse primer comprising SEQ ID NO: 18, SEQ ID NO: 19, SEQ ID NO: 20, and SEQ ID NO: 21, or any active fragments or combinations thereof, and
Primer Set 8 comprises a forward primer comprising SEQ ID NO: 22, SEQ ID NO: 23, SEQ ID NO: 24, or SEQ ID NO: 25, or any active fragments or combinations thereof, and a reverse primer comprising SEQ ID NO: 26, SEQ ID NO: 27, SEQ ID NO: 28, or SEQ ID NO: 29, or any active fragments or combinations thereof.

3.-4. (canceled)

5. A kit for amplifying a V3 region of the HIV env genomic sequence or a portion thereof by nested PCR comprising a set of outer primers consisting of consisting of Primer Set 1, Primer Set 3, Primer Set 5, or Primer Set 7 and a set of inner primers consisting of Primer Set 2, Primer Set 4, Primer Set 6, or Primer Set 8 as defined in claim 2.

6. The kit of claim 5, wherein the forward primer of Primer Set 5 comprises SEQ ID NO: 2 and SEQ ID NO: 3, or any active fragments thereof and the reverse primer of Primer Set 5 comprises SEQ ID NO: 5 and SEQ ID NO: 6, or any active fragments thereof.

7. The kit of claim 5, wherein the forward primer of Primer Set 6 comprises SEQ ID NO: 7 or any active fragment thereof and the reverse primer of Primer Set 6 comprises SEQ ID NO: 9, SEQ ID NO: 10, and SEQ ID NO: 11, or any active fragments thereof.

8.-9. (canceled)

10. A method of amplifying a V3 region of the HIV env genomic sequence or a portion thereof in a sample comprising contacting the sample with a set of outer primers consisting of Primer Set 1, Primer Set 3, Primer Set 5, or Primer Set 7 of claim 2, submitting the resulting mixture to a first nucleic acid amplification reaction, contacting a product of the first nucleic acid amplification reaction with a set of inner primers consisting of Primer Set 2, Primer Set 4, Primer Set 6, or Primer Set 8 of claim 2, and submitting the resulting mixture to a second nucleic acid amplification reaction

11. The method of claim 10, wherein the forward primer of Primer Set 5 comprises SEQ ID NO: 2 and SEQ ID NO: 3, or any active fragments thereof and the reverse primer of Primer Set 5 comprises SEQ ID NO: 5 and SEQ ID NO: 6, or any active fragments thereof.

12. The method of claim 10, wherein the forward primer of Primer Set 6 comprises SEQ ID NO: 7, or any active fragment thereof and the reverse primer of Primer Set 6 comprises SEQ ID NO: 9, SEQ ID NO: 10, and SEQ ID NO: 11, or any active fragments thereof.

13. The method of claim 10, wherein the set of outer primers is Primer Set 1 and the set of inner primers is Primer Set 2.

14. The method of claim 10, wherein the set of outer primers is Primer Set 3 and the set of inner primers is Primer Set 4.

15.-18. (canceled)

19. The method of claim 10 further comprising sequencing a product from the second nucleic acid amplification reaction to detect an HIV env genomic sequence.

20.-28. (canceled)

29. A mixture of two or more of nucleic acids, wherein the mixture comprises a first nucleic acid that encodes all or a portion of a CCR5 coreceptor variant HIV Env protein and a second nucleic acid that encodes all or a portion of a CXCR4 coreceptor variant HIV Env protein, wherein the first and second nucleic acids encode the V3 region of the HIV Env proteins.

30. The mixture of claim 29, wherein the first nucleic acid comprises a sequence that is at least 80%, 85%, 90%, 95% or 99% homologous to the sequence of SEQ ID NO.: 36 or an RNA equivalent thereof and the second nucleic acid comprises sequence that is at least 80%, 85%, 90%, 95% or 99% homologous to the sequence of SEQ ID NO.: 37 or an RNA equivalent thereof.

31. The mixture of claim 29, wherein the first nucleic acid comprises the sequence of SEQ ID NO.: 36 or an RNA equivalent thereof and the second nucleic acid comprises the sequence of SEQ ID NO.: 37 or an RNA equivalent thereof.

32. The mixture of claim 29, wherein the first nucleic acid comprises a sequence that is at least 80%, 85%, 90%, 95% or 99% homologous to the sequence of SEQ ID NO.: 38 or an RNA equivalent thereof and the second nucleic acid comprises sequence that is at least 80%, 85%, 90%, 95% or 99% homologous to the sequence of SEQ ID NO.: 39 or an RNA equivalent thereof.

33. The mixture of claim 29, wherein the first nucleic acid comprises the sequence of SEQ ID NO.: 38 or an RNA equivalent thereof and the second nucleic acid comprises the sequence of SEQ ID NO.: 39 or an RNA equivalent thereof.

34. The mixture of claim 29, wherein the molar concentration of the first nucleic acid is greater than the molar concentration of the second nucleic acid.

35. The mixture of claim 34, wherein the molar concentration of the first nucleic acid is at least 2, 3, 5, 10, 25, 50 or 100 times greater than the molar concentration of the second nucleic acid.

36. The mixture of claim 29, wherein the first and second nucleic acids are RNA transcripts.
